(19) United States
(12) Patent Application Publication
(10) Pub. No.: US 2023/0104823 A1
(43) Pub. Date: Apr. 6, 2023

(54) PROCESS FOR THE PREPARATION OF PURINE DERIVATIVES EXHIBITING CDK INHIBITORY ACTIVITY

(71) Applicant: CYCLACEL LIMITED, LONDON (GB)

(72) Inventors: Benjamin SKEAD, CAMBRIDGE (GB); Derek LONDESBROUGH, HARTLEPOOL (GB); Chris GILL, SEDGEFIELD, STOCKTON (GB); ALEX HUDSON, GATESHEAD TYNE AND WEAR (GB)

(21) Appl. No.: 17/758,102

(22) PCT Filed: Jan. 21, 2021
(86) PCT No.: PCT/GB2021/050134

§ 371 Date: Jun. 28, 2022

(30) Foreign Application Priority Data
Jan. 22, 2020 (GB) 2000901.5

(51) Int. Cl. C07D 473/34

(52) U.S. Cl. CPC C07D 473/34

(57) ABSTRACT

The present invention relates to a process for preparing a compound of formula [I], or a pharmaceutically acceptable salt thereof, said process comprising the steps of: (i) forming a reaction mixture comprising a compound of formula [II] and a compound of formula [III]; (ii) heating said reaction mixture to a temperature of at least about 130° C. to form a compound of formula [I]; (iii) isolating said compound of formula [I] from the mixture and optionally recovering unreacted compound of formula [III]; and (iv) optionally converting said compound of formula [I] into salt form; wherein: R¹ and R² are each independently H, alkyl or haloalkyl; R³ and R⁴ are each independently H, alkyl, haloalkyl or aryl; R⁵ is alkyl, alkenyl, cycloalkyl or cycloalkyl-alkyl, each of which may be optionally substituted with one or more OH groups; R⁶ is selected from cyclopropylamino, cyclopropylmethylamino, cyclobutylamino, cyclobutylmethylamino and where one of X, Y and Z is N and the remainder are CR⁹; R⁷, R⁸ and each R⁹ are independently H, alkyl or haloalkyl, wherein at least one of R⁷, R⁸ and R⁹ is other than H. Further aspects of the invention relate to a process for preparing intermediates of formula [II], and other intermediates useful in the synthesis of compounds of formula [I].

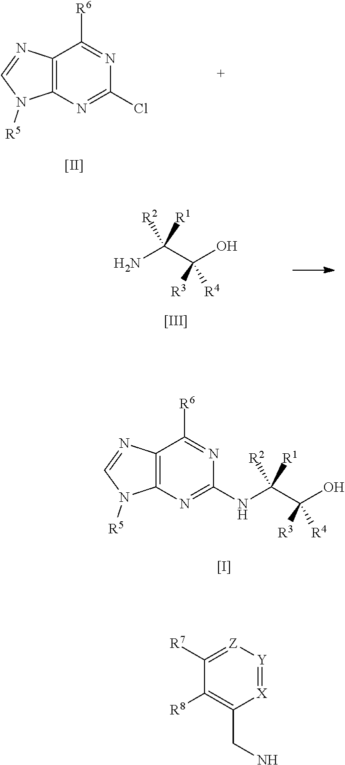

The present invention relates to a process for preparing purine derivatives.

### BACKGROUND TO THE INVENTION

Purine derivatives exhibiting CDK inhibitory activity are disclosed in WO 2008/122767 (Cyclacel Limited; Cancer Research Technology Limited). By way of example, studies have demonstrated that compound [1], having the chemical name (2R,3S)-3-(6-((4,6-dimethylpyridin-3-ylmethylamino)-9-isopropyl-9H-purin-2-ylamino)pentan-2-ol, exhibits potent CDK inhibitory activity and thus has potential therapeutic applications in the treatment of proliferative disorders, immune-mediated and inflammatory disorders, autoimmune and autoimmune-mediated disorders, kidney disorders, cardiovascular disorders, ophthalmic disorders, neurodegenerative disorders, psychiatric disorders, viral disorders, metabolic disorders and respiratory disorders.

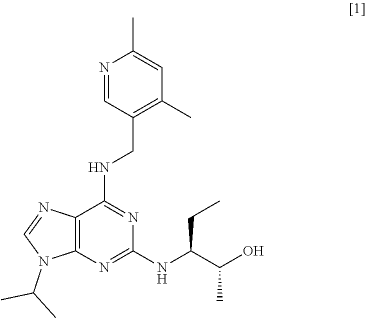 [SMILES: CC[C@H](Nc1nc(NCc2cnc(C)cc2C)c2ncn(C(C)C)c2n1)[C@@H](C)O]

Advantageously, compound [1] displays surprisingly high potency in cellular toxicity studies in a range of different cell lines.

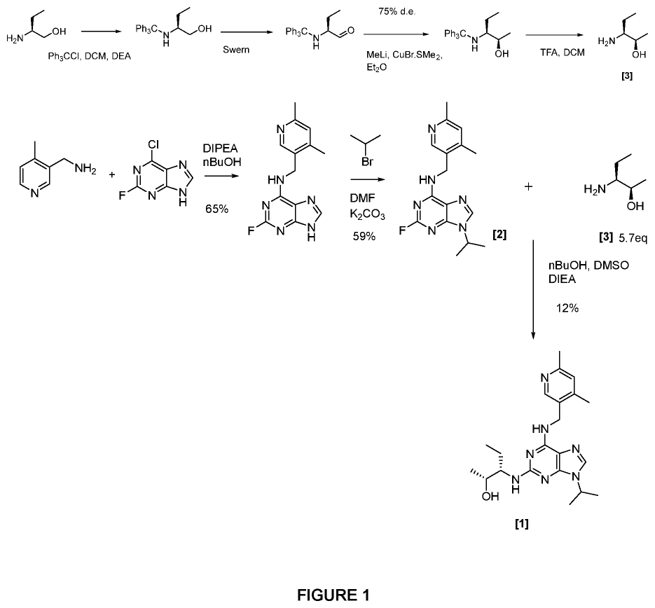

The synthetic preparation of compound [1] was first described in WO 2008/122767. The reaction scheme is shown in FIG. 1. The preparation involved synthesising fluoro-substituted purine derivative [2] and coupling with (2R,3S)-3-aminopentan-2-ol, [3]. The coupling reaction was carried out in ⁿBuOH in the presence of DMSO and DIEA. The reaction required heating at a temperature of 140° C. for 72 hours and yielded only 12% of the desired product. Intermediate compound [3] was prepared via Swern oxidation of (S)-2-(trityl-amino)-butan-1-ol and subsequent reduction with MeLi and CuBr.SMe₂. The resulting intermediate was then treated with trifluoroacetic acid (TFA) to yield compound [3]. However, the MeLi/CuBr.SMe₂ reduction step led to poor stereoselectivity, yielding a product having only 75% diastereomeric excess (d.e.).

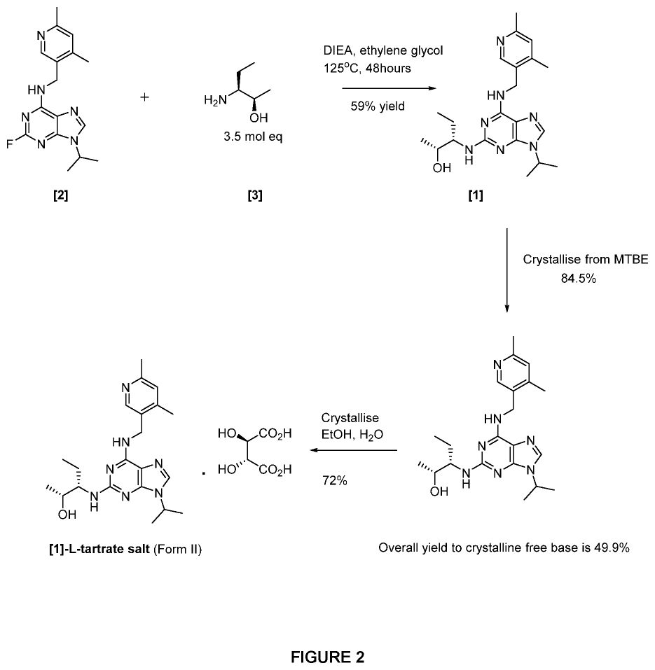

Alternative conditions for the coupling step were disclosed in WO 2011/089401 (Cyclacel Limited), as shown in FIG. 2. These alternative conditions involved reacting compound [2] with compound [3] in DIEA and ethylene glycol at a temperature of 125° C. for 48 hours. This gave rise to a marked improvement in the yield of crude compound [1] (59% cf 12% in WO 2008/122767), which was then crystallised from MTBE to give an overall yield of 49.4%. The crystalline free base material was subsequently converted to the crystalline L-tartrate salt (Form II; also referred to as Form E) in 72% yield by recrystallizing from an ethanol/water mixture.

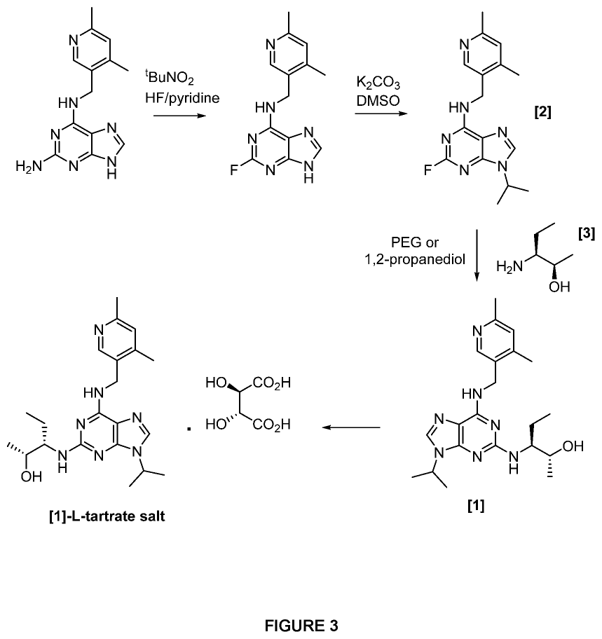

Further modified conditions for the preparation of compound [1] were disclosed in WO 2018/138500 (Cyclacel Limited) as shown in FIG. 3. These conditions involved reacting compound [2] with compound [3] in 1,2-propanediol or polyethylene glycol and heating the reaction mixture to a temperature of at least about 150° C. Advantageously, these alternative coupling conditions led to a marked improvement in the yield. For example, using 1,2-propanediol as the solvent, the overall yield of crystalline free base of compound [1] was shown to be ca. 79% (compared to 59% using the conditions described in WO 2011/089401). WO 2018/138500 also describes an amino precursor to compound [2].

WO 2018/138500 further describes optimised conditions for preparing the crystalline L-tartrate salt of compound [1] comprising refluxing a solution of compound [1] in ethanol and adding dropwise thereto a solution of L-tartaric acid in a mixture of water and ethanol, wherein the ratio of ethanol:water in the final mixture after addition of the L-tartaric acid solution is at least about 15:1. Advantageously, increasing the proportion of ethanol relative to water in the crystallisation step leads to a marked improvement in the yield of the crystalline tartrate salt of compound [1] relative to the yields disclosed in the art (ca. 87% compared with 72% in Example 5.5 of WO 2011/089401).

Finally, WO 2018/138500 describes highly diastereoselective reduction conditions for preparation of amino alcohol [3], which leads to a very high diastereomeric excess (ca. 99%) in the resulting intermediate. This diastereomeric excess far exceeds the levels observed for preparation of such intermediates according to prior art methods; see for example, WO 2003/002565 (Cyclacel Limited) or WO 2008/122767 (Cyclacel Limited; Cancer Research Technology Limited).

The present invention seeks to provide an alternative synthetic preparation for CDK inhibitors such as compound [1]. More specifically, but not exclusively, the present invention seeks to provide a synthetic route which is suitable for scale up and/or which gives rise to one or more of: improved ease of preparation, fewer synthetic steps, reduced amounts of/fewer side products, and/or reduced amounts of reagents (particularly harmful and highly corrosive reagents), whilst at the same time maintaining acceptable yields, purity and stereoselectivity.

### STATEMENT OF INVENTION

A first aspect of the invention relates to a process for preparing a compound of formula [I], or a pharmaceutically acceptable salt thereof,

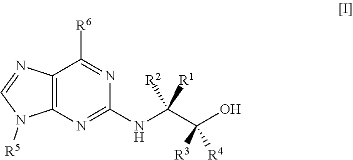

wherein:
R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and

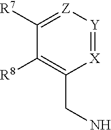

where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of:

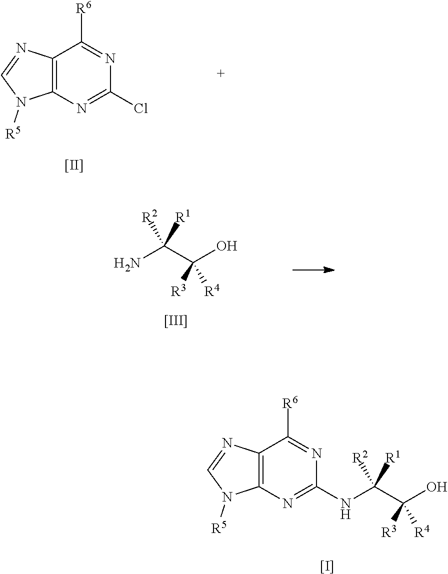

(i) forming a reaction mixture comprising a compound of formula [II], and a compound of formula [III];
(ii) heating said reaction mixture to a temperature of at least about 130° C. to form a compound of formula [I];
(iii) optionally isolating said compound of formula [I] from the mixture and optionally recovering unreacted compound of formula [III]; and
(iv) optionally converting said compound of formula [I] into salt form.

Advantageously, the above-described process involves coupling a 2-chloropurine intermediate [II] with amino alcohol [III]. This contrasts with prior art methods that proceed via the corresponding 2-fluoropurine intermediate, where the 2-fluoropurine intermediate itself was prepared by a fluorination step using the extremely hazardous reagent hydrogen fluoride. The use of a 2-chloropurine intermediate is therefore particularly beneficial in the context of developing a synthetic process suitable for scale-up; firstly, it removes the need for an additional synthetic step, and secondly, and it completely avoids the use of hydrogen fluoride.

A second aspect of the invention relates to a process for preparing a compound of formula [I], or a pharmaceutically acceptable salt thereof,

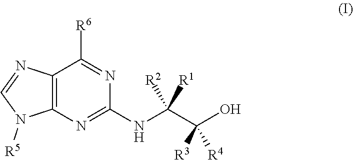

wherein R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and

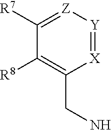

where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of:

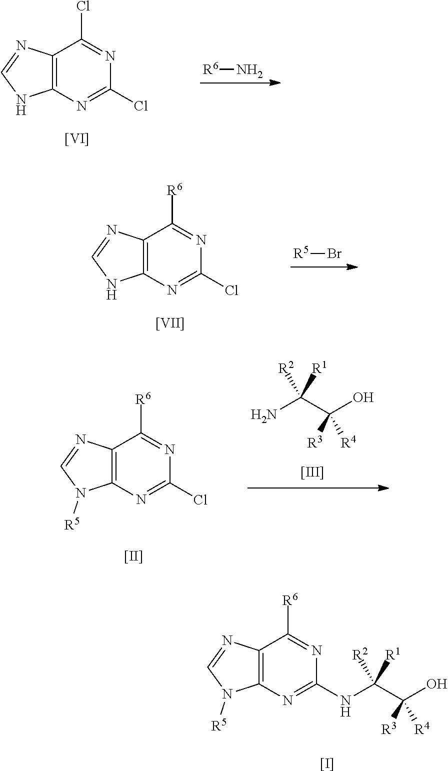

(a) treating a compound of formula [VI] with R⁶—NH₂ or a salt thereof to form a compound of formula [VII];
(b) treating said compound of formula [VII] with R⁵Br to form a compound of formula [II];
(c) forming a reaction mixture comprising a compound of formula [II], and a compound of formula [III] and heating said reaction mixture to a temperature of at least about 130° C. to form a compound of formula [I];
(d) optionally isolating said compound of formula [I] from the mixture and optionally recovering unreacted compound of formula [III]; and
(e) optionally converting said compound of formula [I] into salt form.

A third aspect of the invention relates to a process for preparing a compound of formula [1], or a pharmaceutically acceptable salt thereof, said process comprising the steps of:

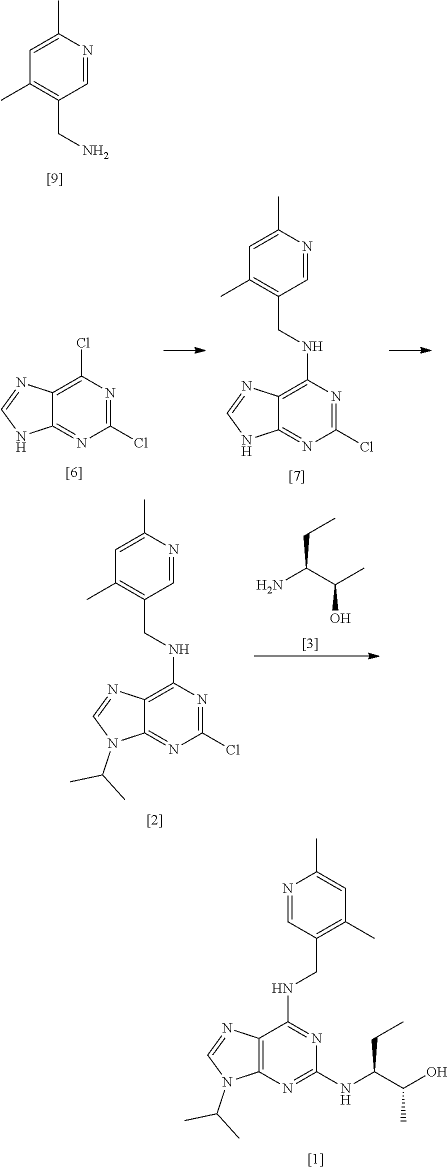

(a) treating a compound of formula [6] with a compound of formula [9], or a salt thereof, to form a compound of formula [7];
(b) treating said compound of formula [7] with isopropyl bromide to form a compound of formula [2];
(c) forming a reaction mixture comprising a compound of formula [2], and compound of formula [3] and heating said reaction mixture to a temperature of at least about 130° C. to form a compound of formula [1];
(d) optionally isolating said compound of formula [1] from the mixture and optionally recovering unreacted compound of formula [3]; and
(e) optionally converting said compound of formula [1] into salt form.

A fourth aspect of the invention relates to a compound of formula [2]:

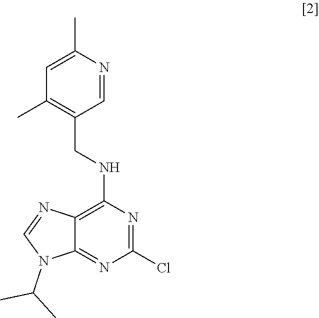 [SMILES: Cc1cc(C)c(CNc2nc(Cl)nc3c2ncn3C(C)C)cn1]

or a salt thereof. Compound [2] is a useful intermediate in the synthesis of compound [1].

### DETAILED DESCRIPTION

### Process for Preparing Compounds of Formula [I]

The present invention provides a new procedure for the synthesis of compounds of general formula [I], and salts thereof, and in particular, the specific compound [1]. Advantageously, the presently claimed process avoids the use of the extremely hazardous reagent hydrogen fluoride which is a particular benefit in the context of developing a synthetic process suitable for scale-up. Furthermore, the presently claimed process involves fewer synthetic steps than prior art methods described to date.

As mentioned, a first aspect of the invention relates to a process for preparing a compound of formula [I], or a pharmaceutically acceptable salt thereof, said process comprising the steps of:
(i) forming a reaction mixture comprising a compound of formula [II], and a compound of formula [III];
(ii) heating said reaction mixture to a temperature of at least about 130° C. to form a compound of formula [I];
(iii) optionally isolating said compound of formula [I] from the mixture and optionally recovering unreacted compound of formula [III]; and
(iv) optionally converting said compound of formula [I] into salt form.

Advantageously, the reaction between compound [II] and compound [III] does not require the presence of a base. Nor does the reaction require the presence of a separate solvent; instead the reaction can take place in neat amino alcohol [III] (the mixture of [II] and [III] forming a slurry). This minimises the amount of additional reagents required, which is again beneficial for scale-up purposes.

Thus, in one preferred embodiment, the reaction between compound [II] and compound [III] takes place in the absence of a solvent, i.e. compound [III] forms a solution with compound [II] and no additional solvent is required.

As used herein, the term “alkyl” includes both saturated straight chain and branched alkyl groups. Preferably, the alkyl group is a C₁₋₂₀ alkyl group, more preferably a C₁₋₁₅, more preferably still a C₁₋₁₂ alkyl group, more preferably still, a C₁₋₆alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “cycloalkyl” refers to a cyclic alkyl group. Preferably, the cycloalkyl group is a C₃₋₁₂ cycloalkyl group.

As used herein, the term “cycloalkyl-alkyl” refers to a group having both cycloalkyl and alkyl functionalities.

As used herein, the term “alkenyl” includes both straight chain and branched alkenyl groups. Preferably, the alkenyl group is a C₂₋₂₀ alkyl group, more preferably a C₂₋₁₅, more preferably still a C₂₋₁₂ alkyl group, more preferably still, a C₂₋₆ alkyl group, more preferably a C₂₋₃ alkyl group.

“Halo” is defined herein as chloro, fluoro, bromo or iodo.

As used herein, the term “aryl” refers to a C₆₋₁₂ aromatic group, which may be benzocondensed, for example, phenyl or naphthyl. Preferably, the aryl group is a phenyl group.

In one preferred embodiment, the process comprises recovering unreacted compound of formula [III]. Preferably, the unreacted compound of formula [III] is recovered by distillation, more preferably by fractional distillation of the crude reaction mixture. Preferably, the crude reaction mixture is fractionally distilled in vacuo at 30 to 50 mBar and at a temperature of from about 80 to about 170° C. In one highly preferred embodiment, the unreacted amino alcohol [III] is recovered in a work up procedure which comprises charging the reaction mixture with a suitable solvent (e.g. a polyethylene glycol, preferably PEG300 or PEG400, more preferably PEG400), and then adding a base (preferably aqueous NaOH). Unreacted amino alcohol can then be recovered by vacuum distillation.

In another preferred embodiment, the process proceeds without the step of recovering unreacted compound of formula [III].

In one preferred embodiment of the invention, the process comprises the steps of:
(i) forming a reaction mixture comprising a compound of formula [II], and a compound of formula [III];
(ii) heating said reaction mixture to a temperature of at least about 130° C. to form a compound of formula [I];
(iii) isolating said compound of formula [I] from the mixture and optionally recovering unreacted compound of formula [III]; and
(iv) optionally converting said compound of formula [I] into salt form.

In one preferred embodiment of the invention, the process comprises the steps of:
(i) forming a reaction mixture comprising a compound of formula [II], and a compound of formula [III];
(ii) heating said reaction mixture to a temperature of at least about 130° C. to form a compound of formula [I];
(iii) separating said compound of formula [I] from unreacted compound of formula [III]; and
(iv) optionally converting said compound of formula [I] into salt form.

In one preferred embodiment, the compound of formula [I] is isolated from the reaction mixture by acidifying any unreacted compound of formula [III] with aqueous acid and extracting into a suitable organic solvent (preferably ethyl acetate or butyl acetate, more preferably ethyl acetate). In a more preferred embodiment, step (iii) comprises extracting the reaction mixture from step (ii) into aqueous HCl and an organic solvent, separating the organic phase and concentrating the filtrate. In one preferred embodiment, the compound of formula [I] is then converted to salt form, i.e. without further purification or crystallization of the free base material.

In another preferred embodiment, the compound of formula [I] is further purified by crystallization. In one preferred embodiment of the invention, step (iii) further comprises the step of crystallizing compound [I] from a suitable solvent. Crystalline compound [I] can then be converted to salt form as described below. In one preferred embodiment, compound [I] is crystallised from a solvent selected from ethyl acetate, isopropyl acetate, n-butyl acetate, isobutyl acetate and methyl isobutyl ketone (MIBK) or mixtures of two or more thereof. Preferably the solvent is anhydrous. Preferably, the solvent (or mixture of solvents) is heated to a temperature of at least 50° C.

In one preferred embodiment, compound [I] is crystallised from n-butyl acetate.

In one preferred embodiment, compound [I] is crystallised from ethyl acetate.

In one preferred embodiment, compound [I] is crystallised from isopropyl acetate.

In one preferred embodiment, one or more alkanes (for example, hexane, heptane or the like) are added to the crystallisation solvent as an antisolvent to increase yields of crystalline compound [I]. In one preferred embodiment, the solvent is a mixture of ethyl acetate and heptane.

In another preferred embodiment, the solvent is a mixture of isopropyl acetate and heptane.

In one particularly preferred embodiment, the solvent is a mixture of n-butyl acetate and heptane.

In another preferred embodiment, the process comprises the steps of:
forming a reaction mixture comprising a compound of formula [II], and a compound of formula [III];
heating said reaction mixture to a temperature of at least about 130° C. to form a compound of formula [I]; and
optionally converting said compound of formula [I] into salt form.

In another preferred embodiment, the process comprises the steps of:
forming a reaction mixture comprising a compound of formula [II], and a compound of formula [III];
heating said reaction mixture to a temperature of at least about 130° C. to form a compound of formula [I]; and
converting said compound of formula [I] into salt form.

In one preferred embodiment, after heating the reaction mixture to a temperature of at least about 130° C. to form a compound of formula [I], the mixture is cooled (preferably to a temperature of about 60° C.) and the remaining compound [III] is acidified with a suitable acid (e.g. 1 mol equivalent of HCl). Compound [I] is then extracted into a suitable organic solvent (preferably ethyl acetate) and washed with water. In a preferred embodiment, the organic phase is then concentrated by distillation and charged with ethanol. Distillation is then continued until the organic solvent (e.g. ethyl acetate) is removed, i.e. there is a “solvent swap” to ethanol. The ethanol solution of compound [I] can then be converted to salt form as described below.

In one preferred embodiment of the invention, the process comprises converting said compound of formula [I] into the corresponding L-tartrate salt.

In one preferred embodiment, one of R¹ and R² is H and the other is alkyl.

More preferably, one of R¹ and R² is H and the other is methyl, ethyl or isopropyl.

Even more preferably, R¹ is alkyl, more preferably ethyl, and R² is H.

In one preferred embodiment, R³ and R⁴ are each independently H, alkyl, haloalkyl or aryl, and wherein at least one of R³ and R⁴ is other than H.

In one preferred embodiment, one of R³ and R⁴ is H and the other is alkyl or haloalkyl.

In one preferred embodiment, R³ is H and R⁴ is alkyl or haloalkyl.

In one preferred embodiment, R³ is H and R⁴ is methyl.

In one preferred embodiment, R¹ and R⁴ are each independently alkyl, and R² and R³ are both H. Preferably, R² and R³ are both H, R¹ is ethyl and R⁴ is Me.

In one preferred embodiment, R⁶ is:

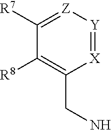

In one preferred embodiment, Y is N and X and Z are both CR⁹.

In one preferred embodiment, Y is N; preferably for this embodiment:

X is CH, Z is C-Me and R⁷ is H and R⁸ is Me; or
X is CH, Z is C-Me and R⁷ and R⁸ are both H; or
X is CH, Z is C—CF₃ and R⁷ and R⁸ are both H.

More preferably, Y is N, X is CH, Z is C-Me, R⁷ is H and R⁸ is Me.

In another preferred embodiment, X is N. Preferably for this embodiment:

Y is C-Me, Z is CH and R⁷ and R⁸ are both H; or
Y and Z are CH, R⁷ is H and R⁸ is Me.

In another preferred embodiment Z is N. Preferably, for this embodiment, X is CH, Y is C-Me, R⁷ is Me and R⁸ is H.

In another preferred embodiment, R⁶ is cyclopropylamino, cyclopropylmethylamino, cyclobutylamino or cyclobutylmethylamino.

In one preferred embodiment, R⁵ is isopropyl or isopropenyl, more preferably, isopropyl.

In one highly preferred embodiment, the compound of formula [I] is selected from the following:
2R,3S-3-(6-((4,6-Dimethylpyridin-3-ylmethylamino)-9-isopropyl-9H-purin-2-ylamino)pentan-2-ol
2R,3S-3-(6-Cyclopropylamino)-9-isopropyl-9H-purin-2-ylamino)pentan-2-ol
2R,3S-3-(6-(Cyclopropyl methylamino)-9-isopropyl-9H-purine-2-ylamino)pentan-2-ol
2R,3S-3-(6-(Cyclobutylamino)-9-isopropyl-9H-purin-2-ylamino)pentan-2-ol
2R,3S-3-(9-Isopropyl-6-(2,6-dimethylpyridine-4-ylmethylamino)-9H-purin-2-ylamino)pentan-2-ol
2R,3S-3-(9-Isopropyl-6-((6-(trifluoromethyl)pyridine-3-yl)methylamino)-9H-purin-2ylamino)pentan-2-ol
2R,3S-3-(9-Isopropyl-6-((6-methyl pyridin-2-yl)methylamino)-9H-purin ylamino)pentan-2-ol
2R,3S-3-(9-Isopropyl-6-((3-methylpyridin-2-yl)methylamino)-9H-purin ylamino)pentan-2-ol
1,1,1-Trifluoro-3-(9-isopropyl-6-((6-(trifluoromethyl)pyridin-3-yl)methylamino)-9H-2-ylamino)pentan-2-ol

In one preferred embodiment:

the compound of general formula [I] is compound [1];
the compound of general formula [II] is compound [2]; and
the compound of general formula [III] is compound [3];
i.e. the invention relates to a process which comprises the steps of:

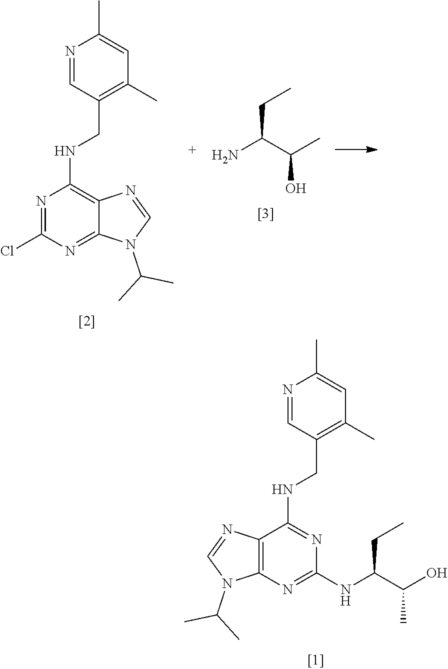

(i) forming a reaction mixture comprising a compound of formula [2], and a compound of formula [3];
(ii) heating said reaction mixture to a temperature of at least about 130° C. to form a compound of formula [1];
(iii) optionally isolating said compound of formula [1] from the mixture and optionally recovering unreacted compound of formula [3]; and
(iv) optionally converting said compound of formula [1] into salt form.

In one preferred embodiment, the reaction mixture in step (ii) is heated to a temperature of from about 135° C. to about 220° C., more preferably from about 135° C. to about 200° C. Where the reaction mixture is heated to higher temperatures, for example, in excess of 180° C., the reaction is preferably carried out in a sealed system, for example, an autoclave. In one preferred embodiment, the reaction mixture in step (ii) is heated to a temperature of from about 135° C. to about 175° C. In one preferred embodiment, the reaction mixture is heated to a temperature of from about 135° C. to about 160° C., more preferably from about 135° C. to about 155° C., more preferably from about 135° C. to about 150° C., even more preferably from about 135° C. to about 140° C. In another preferred embodiment, the reaction mixture is heated to a temperature of from about 150° C. to about 175° C., more preferably, from about 150° C. to about 170° C., or about 150° C. to about 160° C. or about 155° C. to about 160° C. In one preferred embodiment, the reaction mixture in step (ii) is heated to a temperature of at least 140° C. In another preferred embodiment the reaction mixture in step (ii) is heated to a temperature of from about 140° C. to about 160° C., more preferably, from about 140° C. to about 155° C. or from about 140° C. to about 150° C.

In another preferred embodiment, the reaction mixture in step (ii) is heated to a temperature of from about 140° C. to about 220° C., more preferably from about 140° C. to about 200° C., more preferably from about 160° C. to about 200° C. or even more preferably from about 180° C. to about 200° C.

In one particularly preferred embodiment, the reaction mixture is heated to a temperature of about 150° C.

In one preferred embodiment, the reaction mixture in step (ii) is heated for a period of at least 12 hours. In a more preferred embodiment, the reaction mixture in step (ii) is heated for a period of at least 24 hours. In another preferred embodiment, the reaction mixture is heated for a period of at least 48 hours. In another preferred embodiment, the reaction mixture is heated for a period of at least 72 hours. In one highly preferred embodiment, the reaction mixture is heated for a period of about 24 hours. In another highly preferred embodiment, the reaction mixture is heated for a period of about 48 hours. In another highly preferred embodiment, the reaction mixture is heated for a period of about 72 hours.

In another preferred embodiment, the reaction mixture in step (ii) is heated for a period of from about 24 to about 96 hours, more preferably, from about 24 to about 72 hours, or from about 24 to about 48 hours. In another preferred embodiment, the reaction mixture is heated for a period of from about 48 to about 96 hours, more preferably, from about 48 to about 72 hours.

In one preferred embodiment, the reaction between compound [II] and compound [III] takes place in the absence of a solvent, i.e. compound [III] forms a solution with compound [II] and no additional solvent is required.

In one preferred embodiment, the reaction mixture in step (ii) comprises from about 4 to about 7 mole equivalents of compound [III] relative to compound [II]. In a more preferred embodiment, the reaction mixture comprises from about 5 to about 7 mole equivalents of compound [III] relative to compound [II]. More preferably, the reaction mixture comprises from about 5 to about 6 or about 5 to about 5.5 mole equivalents of compound [III] relative to compound [II]. Even more preferably, the reaction mixture comprises about 5 mole equivalents of compound [III] relative to compound [II].

In one preferred embodiment, the reaction between compound [1] and compound [3] takes place in the absence of a solvent, i.e. compound [3] forms a solution with compound [2] and no additional solvent is required.

In one particularly preferred embodiment, in the context of preparing a compound of formula [1], the reaction mixture comprises from about 4 to about 7 mole equivalents of compound [3] relative to compound [2]. In a more preferred embodiment, the reaction mixture comprises from about 5 to about 7 mole equivalents of compound [3] relative to compound [2]. More preferably, the reaction mixture comprises from about 5 to about 6 or about 5 to about 5.5 mole equivalents of compound [3] relative to compound [2]. Even more preferably, the reaction mixture comprises about 5 mole equivalents of compound [3] relative to compound [2].

In one preferred embodiment, the process comprises the steps of:
(i) forming a reaction mixture comprising a compound of formula [2], and a compound of formula [3];
(ii) heating said reaction mixture to a temperature of at least about 130° C. to form a compound of formula [1];
(iii) isolating said compound of formula [1] from the mixture and optionally recovering unreacted compound of formula [3]; and
(iv) optionally converting said compound of formula [1] into salt form.

In one preferred embodiment of the invention, the process comprises the steps of:
(i) forming a reaction mixture comprising a compound of formula [2], and a compound of formula [3];
(ii) heating said reaction mixture to a temperature of at least about 130° C. to form a compound of formula [1];
(iii) separating said compound of formula [1] from unreacted compound of formula [3]; and
(iv) optionally converting said compound of formula [1] into salt form.

In one preferred embodiment, step (iii) comprises extracting the reaction mixture from step (ii) into aqueous HCl (to neutralise any remaining unreacted amino alcohol and allow extraction of the HCl salt into the aqueous phase) and n-butyl acetate, separating the n-butyl acetate phase and drying with a drying agent, filtering and concentrating the filtrate to reduce its volume. Suitable drying agents (for example, magnesium sulfate) will be familiar to the skilled person in the art. The reduced volume organic phase is then heated under nitrogen, seeded with the product (e.g. compound [1]), and gradually cooled before charging with heptane. The product is then filtered, washed (for example, with a mixture of 2:1 n-butyl acetate/heptane) and dried in vacuo. Preferably, the seeding takes place using the crystalline free base form of compound [1] designated as Form A and described in WO 2011/089401 (Cyclacel Limited; see in particular, Example 1), the contents of which are hereby incorporated by reference.

In one highly preferred embodiment, the unreacted amino alcohol [III] or [3] is recovered by a work up procedure which comprises charging the reaction mixture with a suitable solvent (e.g. a polyethylene glycol, preferably PEG300 or PEG400, more preferably PEG400), and then adding a base (preferably aqueous NaOH). Any unreacted amino alcohol is then recovered by vacuum distillation. The remaining reaction mixture is then charged with n-butyl acetate and brine, and the organic phase dried with a drying agent (e.g. magnesium sulfate). The reduced volume organic phase is then heated under nitrogen, seeded with the product (e.g. compound [1]), and gradually cooled before charging with heptane. The product is then filtered, washed (for example, with a mixture of 2:1 n-butyl acetate/heptane) and dried in vacuo. Advantageously, this method allows up to 4 equivalents of the amino alcohol to be recovered.

In another preferred embodiment, the process does not comprise the step of recovering unreacted compound of formula [III] or [3].

In one preferred embodiment, the process of the invention comprises the further step of preparing a compound of formula [II] by:

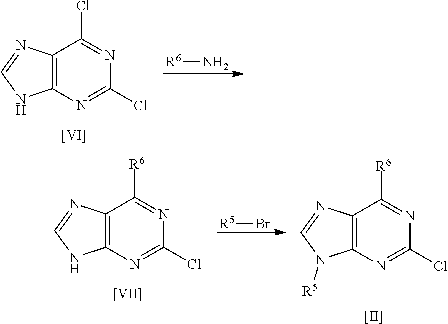

(i) treating a compound of formula [VI] with R⁶—NH₂, or a salt thereof, to form a compound of formula [VII]; and
(ii) treating said compound of formula [VII] with R⁵Br to form a compound of formula [II],
   where R⁵ and R⁶ are as defined above.

As used throughout the term “treating” means bringing two or more components into contact in an appropriate environment (e.g. reaction vessel) and under appropriate conditions (e.g. temperature, concentration, pressure) to allow a reaction to take place between the components.

In one preferred embodiment, the compound of formula [VII] formed in step (i) is isolated prior to step (ii). In a further preferred embodiment, the compound of formula [VII] formed in step (i) is isolated and purified prior to step (ii).

In one preferred embodiment, the compound of formula [II] formed in step (ii) is isolated prior to reacting with amino alcohol [III]. In a further preferred embodiment, the compound of formula [II] formed in step (i) is isolated and purified prior to reacting with amino alcohol [III].

In an alternative preferred embodiment, the process of the invention comprises the further step of preparing a compound of formula [II] by:

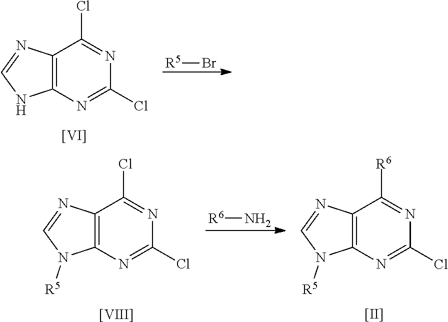

(i) treating a compound of formula [VI] with R⁵Br to form a compound of formula [VIII]; and
(ii) treating said compound of formula [VIII] with R⁶—NH₂, or a salt thereof, to form a compound of formula [II];
   where R⁵ and R⁶ are as defined above.

In one preferred embodiment, the compound of formula [VIII] formed in step (i) is isolated prior to step (ii). In a further preferred embodiment, the compound of formula [VIII] formed in step (i) is isolated and purified prior to step (ii).

In one preferred embodiment, the compound of formula [II] formed in step (ii) is isolated prior to reacting with amino alcohol [III]. In a further preferred embodiment, the compound of formula [II] formed in step (i) is isolated and purified prior to reacting with amino alcohol [III].

In one preferred embodiment, the amine R⁶—NH₂ is in the form of a salt, preferably the hydrochloride salt, even more preferably, the dihydrochloride salt, R⁶—NH₂0.2HCl.

In one preferred embodiment, the reaction with R⁶—NH₂, or salt thereof, is carried out at a temperature of at least 100° C., more preferably, at least 110° C., even more preferably at least 115° C. Preferably the reaction mixture is maintained at this temperature for at least 12 hours, more preferably, at least 18 hours, even more preferably, at least 24 hours. Preferably, the reaction is carried out under nitrogen or another inert gas. Preferably, the reaction takes place in a solvent, more preferably nBuOH. Preferably, the reaction takes place in the presence of a base. Preferably, the base is a tertiary aliphatic amine base. More preferably, the base is selected from N,N-diisopropyl-ethylamine (DIEA), tri-^{N}propylamine, and tri-^{N}butylamine. More preferably still, the base is N,N-diisopropylethylamine (DIEA). Preferably, the base is present in 3-5 mole equivalents relative to the compound of formula [VI] (or formula [VIII]), more preferably, 3 to 4 mole equivalents, even more preferably, about 3.5 mole equivalents. Preferably, the compound R⁶—NH₂, or salt thereof, is present in an amount of about 1 mole equivalent relative to the compound of formula [VI] (or formula [VIII]). After maintaining the reaction temperature for the aforementioned time period, the reaction mixture is then allowed to cool to room temperature, filtered and the resulting solid washed, for example, with tertiary butyl methyl ether (TBME), and dried in vacuo.

In one preferred embodiment, the reaction with R⁵—Br is carried out at a temperature of at least 55° C., more preferably, at least 60° C., even more preferably at least 65° C. Preferably the reaction mixture is maintained at this temperature for at least 30 minutes, more preferably, at least 45 minutes, even more preferably, at least 60 minutes. Preferably, the reaction is carried out under nitrogen or another inert gas. Preferably, the reaction takes place in a solvent, more preferably DMSO. Preferably, the reaction takes place in the presence of a base, more preferably, K₂CO₃. Preferably, the compound R⁵—Br is present in an amount of at least 3 mole equivalents relative to the compound of formula [VI] (or formula [VII]). Preferably, the compound R⁵—Br is present in an amount of 3 to 5 mole equivalents relative to the compound of formula [VI] (or formula [VII]), more preferably 3 to 4 mole equivalents, even more preferably about 3 mole equivalents. After maintaining the reaction temperature for the aforementioned time period, the reaction mixture is then allowed to cool to room temperature, and extracted, for example, with water/ethyl acetate. The organic phase is then concentrated and purified, for example, using a SiO₂ plug or silica chromatography.

In one preferred embodiment of the invention, the process comprises the further step of preparing a compound of formula [2] by:

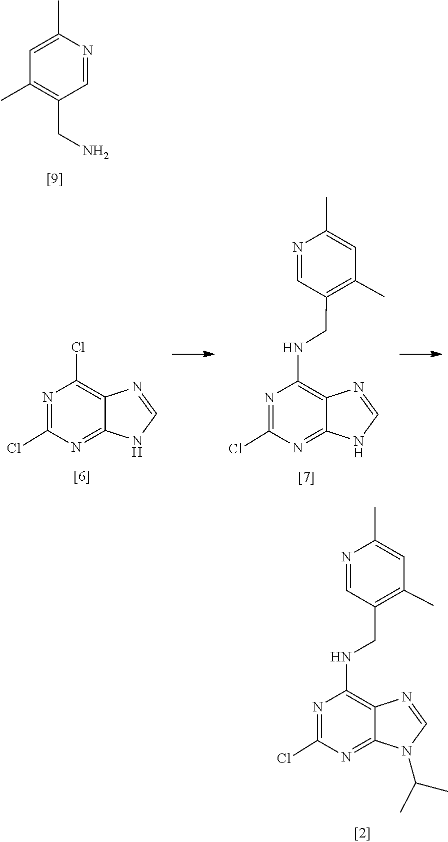

(i) treating a compound of formula [6] with a compound of formula [9], or a salt thereof, to form a compound of formula [7]; and
(ii) treating said compound of formula [7] with isopropyl bromide to form a compound of formula compound [2].

In one preferred embodiment, the compound of formula [7] formed in step (i) is isolated prior to step (ii). In a further preferred embodiment, the compound of formula [7] formed in step (i) is isolated and purified prior to step (ii).

In one preferred embodiment, the compound of formula [2] formed in step (ii) is isolated prior to reacting with amino alcohol [3]. In a further preferred embodiment, the compound of formula [2] formed in step (i) is isolated and purified prior to reacting with amino alcohol [3].

In another preferred embodiment of the invention, the process comprises the further step of preparing a compound of formula [2] by:

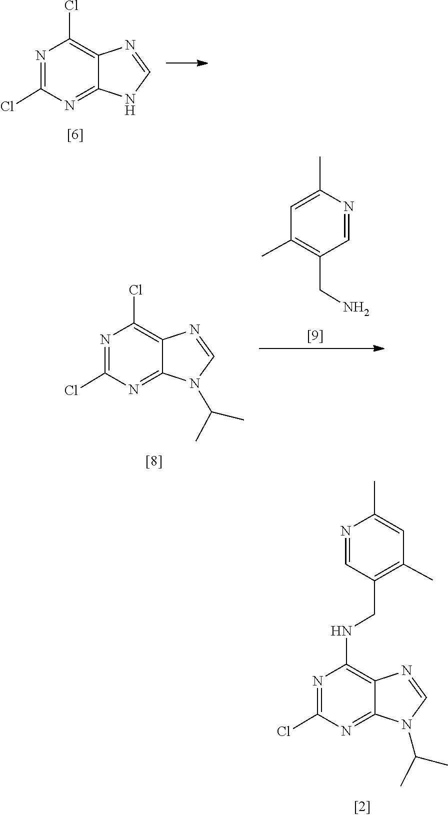

(i) treating a compound of formula [6] with isopropyl bromide to form a compound of formula compound [8]; and
(ii) treating said compound of formula [8] with a compound of formula [9], or a salt thereof, to form a compound of formula [2].

In one preferred embodiment, the compound of formula [8] formed in step (i) is isolated prior to step (ii). In a more preferred embodiment, the compound of formula [8] formed in step (i) is isolated and purified prior to step (ii).

In one preferred embodiment, the compound of formula [2] formed in step (ii) is isolated prior to reacting with amino alcohol [3]. In a more preferred embodiment, the compound of formula [2] formed in step (ii) is isolated and purified prior to reacting with amino alcohol [3].

In one preferred embodiment, compound [9] is in the form of a salt, more preferably a hydrochloride salt, even more preferably, the dihydrochloride salt.

In one preferred embodiment, the reaction with compound [9] or salt thereof is carried out at a temperature of at least 100° C., more preferably, at least 110° C., even more preferably at least 115° C. Preferably the reaction mixture is maintained at this temperature for at least 12 hours, more preferably, at least 18 hours, even more preferably, at least 24 hours. Preferably, the reaction is carried out under nitrogen or another inert gas. Preferably, the reaction takes place in a solvent, more preferably nBuOH. Preferably, the reaction takes place in the presence of a base. Preferably, the base is a tertiary aliphatic amine base. More preferably, the base is selected from N,N-diisopropylethylamine (DIEA), tri-^{N}propylamine, and tri-^{N}butylamine. More preferably still, the base is N,N-diisopropylethylamine (DIEA). Preferably, the base is present in 3-5 mole equivalents relative to the compound of formula [6] (or formula [8]), more preferably, 3 to 4 mole equivalents, even more preferably, about 3.5 mole equivalents.

Preferably, the compound [9], or salt thereof, is present in an amount of about 1 mole equivalent relative to the compound of formula [6] (or formula [8]). After maintaining the reaction temperature for the aforementioned time period, the reaction mixture is then allowed to cool to room temperature, filtered and the resulting solid washed, for example, with tertiary butyl methyl ether (TBME), and dried in vacuo.

In one preferred embodiment, the reaction with isopropyl bromide (2-bromopropane) is carried out at a temperature of at least 55° C., more preferably, at least 60° C., even more preferably at least 65° C. Preferably the reaction mixture is maintained at this temperature for at least 30 minutes, more preferably, at least 45 minutes, even more preferably, at least 60 minutes. Preferably, the reaction is carried out under nitrogen or another inert gas. Preferably, the reaction takes place in a solvent, more preferably DMSO. Preferably, the reaction takes place in the presence of a base, more preferably, K₂CO₃. Preferably, the isopropyl bromide is present in an amount of at least 3 mole equivalents relative to the compound of formula [6] (or formula [7]). Preferably, the isopropyl bromide is present in an amount of 3 to 5 mole equivalents relative to the compound of formula [6] (or formula [7]), more preferably 3 to 4 mole equivalents, even more preferably about 3 mole equivalents. After maintaining the reaction temperature for the aforementioned time period, the reaction mixture is then allowed to cool to room temperature, and extracted with water/ethyl acetate. The organic phase is then concentrated and purified, for example, using a SiO₂ plug or silica chromatography.

In one preferred embodiment, the compound of formula [1] is isolated from the reaction mixture by acidifying any unreacted compound of formula [3] with aqueous acid and extracting into a suitable organic solvent (preferably ethyl acetate or butyl acetate, more preferably ethyl acetate). In a more preferred embodiment, step (iii) comprises extracting the reaction mixture from step (ii) into aqueous HCl and an organic solvent, separating the organic phase and concentrating the filtrate. In one preferred embodiment, the compound of formula [1] is then converted to salt form, i.e. without further purification or crystallization of the free base material.

In another preferred embodiment, the compound of formula [1] is further purified by crystallization. Thus, in one preferred embodiment, in the context of preparing compound [1], step (iii) further comprises the step of crystallizing compound [1] from a suitable solvent. Crystalline compound [1] can then be converted to salt form as described below. In one preferred embodiment, compound [1] is crystallised from a solvent selected from ethyl acetate, isopropyl acetate, n-butyl acetate, isobutyl acetate and methyl isobutyl ketone (MIBK) or mixtures of two or more thereof. Preferably the solvent is anhydrous. Preferably, the solvent (or mixture of solvents) is heated to a temperature of at least 50° C. In one preferred embodiment, one or more alkanes (for example, hexane, heptane or the like) are added to the crystallisation solvent as an antisolvent to increase yields.

In one preferred embodiment, compound [1] is crystallised from n-butyl acetate.

In one particularly preferred embodiment, compound [1] is crystallised from a mixture of n-butyl acetate and heptane.

In one highly preferred embodiment, this step comprises heating a concentrated solution of compound [1] in n-butyl acetate to a temperature of about 70° C., seeding with a crystal of compound [1], cooling the seeded mixture to room temperature, charging the reaction mixture with heptane, and then cooling the seeded mixture to about 0° C. Preferably, the mixture is stirred at this temperature for about 30 minutes and then allowed to cool to room temperature. The seed crystal of compound [1] can be prepared in accordance with the procedures of WO 2011/089401 (see, in particular, Example 1) or WO 2018/138500, the contents of which are hereby incorporated by reference. The resulting product can then be filtered and washed, for example, with 2:1 n-butyl acetate/heptane (preferably cold), and dried in vacuo.

In another preferred embodiment, the process comprises the steps of:
forming a reaction mixture comprising a compound of formula [2], and a compound of formula [3];
heating said reaction mixture to a temperature of at least about 130° C. to form a compound of formula [1]; and
optionally converting said compound of formula [1] into salt form.

In one preferred embodiment, the process comprises the step of converting said compound of formula [1] into salt form, i.e. the process comprises the steps of:
forming a reaction mixture comprising a compound of formula [2], and a compound of formula [3];
heating said reaction mixture to a temperature of at least about 130° C. to form a compound of formula [1]; and
converting said compound of formula [1] into salt form.

In one preferred embodiment, after heating the reaction mixture to a temperature of at least about 130° C. to form a compound of formula [1], the mixture is cooled (preferably to a temperature of about 60° C.) and the remaining compound [3] is acidified with a suitable acid (e.g. 1 mol equivalent of HCl). Compound [1] is then extracted into a suitable organic solvent (preferably ethyl acetate) and washed with water. In one preferred embodiment, the organic phase is then concentrated by distillation and charged with ethanol. Distillation is then continued until the organic solvent (e.g. ethyl acetate) is removed, i.e. there is a “solvent swap” to ethanol. The ethanol solution of compound [1] can then be converted to salt form as described below.

### Salt Formation

In one embodiment, the process comprises the step of converting the compound of formula [I] or [1] into the form of a pharmaceutically acceptable salt form, i.e. step (iv) is present.

Pharmaceutically acceptable salts include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

In one highly preferred embodiment of the invention, the process comprises converting the compound of formula [I] or [1] into the corresponding tartrate salt.

In one particularly preferred embodiment, the process comprises the step of converting said compound of formula [I] or [1] into the L-tartrate salt, more preferably the L-tartrate salt in crystalline form. Even more preferably, the L-tartrate salt is crystalline form II (corresponding to Form E as described in WO 2011/089401) and can be prepared by the methods described therein (see Example 5, in particular Example 5.5).

Thus, in one preferred embodiment, the process comprises refluxing the product isolated in step (iii) (i.e. the compound of formula [I] or [1]) in ethanol and adding dropwise thereto a solution of L-tartaric acid in a mixture of water and ethanol. In one preferred embodiment, this step is carried out on crude compound [I] or [1] without further purification of the free base material by crystallization.

In one particularly preferred embodiment, the ratio of ethanol:water in the final mixture after addition of the L-tartaric acid solution is at least 15:1, more preferably, at least 20:1, more preferably at least 25:1, even more preferably still, at least 30:1. Advantageously, increasing the proportion of ethanol relative to water in the crystallisation step leads to a marked improvement in the yield of the crystalline tartrate salt of compound [1] relative to the yields disclosed in the art (ca. 87% compared with 72% in Example 5.5 of WO 2011/089401).

In one particularly preferred embodiment, the ratio of ethanol:water in the final mixture after addition of the L-tartaric acid solution is about 37.5:1.

In one preferred embodiment, the process comprises maintaining the temperature at 75 to 78° C. during the addition of the solution of L-tartaric acid.

In one preferred embodiment, the crystallisation step further comprises polish filtering the mixture, warming the filtrate to a temperature of about 60 to about 65° C. and seeding with crystalline [1]-L-tartrate form II. Crystalline [1]-L-tartrate form II (also known as Form E) can be prepared in accordance with the teachings of WO 2011/089401, the contents of which are incorporated herein by reference (Cyclacel Limited); see in particular, Example 5.

In one preferred embodiment, the seeded filtrate is stirred at a temperature of about 60 to about 65° C. for at least 1 hour.

In one preferred embodiment, the process further comprises the step of cooling the mixture to a temperature of about 15 to about 20° C. and stirring at that temperature for at least 1 hour to induce crystallisation of compound [1]-L-tartrate. Preferably, the cooling rate is about 5 to about 10° C./hour, more preferably about 10° C./hour.

In one preferred embodiment, the compound [1]-L-tartrate is filtered, washed with ethanol and dried in vacuo.

Advantageously, in the context of the coupling reaction, the use of a high purity intermediate of formula [II] is important in order to obtain the free base compound of formula [I] in sufficient purity to attain the target specification of compound [I]-L-tartrate salt.

In one preferred embodiment, the compound of formula [II] (e.g. compound [2]) is passed through a silica pad, slurried in diethyl ether, filtered and dried prior to step (i).

In one preferred embodiment, the compound of formula [II] (e.g. compound [2]) has a purity of at least 97%, more preferably, at least 97.5% even more preferably, at least 98% by HPLC.

The use of a compound of formula [III] with high disastereromic purity (i.e. a high d.e.) is also important in order to obtain good yields in the coupling reaction with compounds of formula [II]. In particular, using compounds of general formula [III] with high disastereromic purity in the coupling step enables compounds of formula [I] to be prepared in high yield without the need for chromatographic separation from its stereoisomer. Instead, the crude product can simply be isolated and purified by crystallisation as described above, which has obvious advantages in terms of efficiency of scale up.

In one preferred embodiment, the compound of formula [III] (e.g. compound [3]) has a diastereomeric excess of at least 85%, more preferably, at least 90%, even more preferably, at least 95%.

In one highly preferred embodiment, the compound of formula [III] (e.g. compound [3]) has a diastereomeric excess of at least 96, 97, 98 or 99%.

In one preferred embodiment, the compound of formula [III] as defined above, where R₂ is H, is prepared by the steps of:

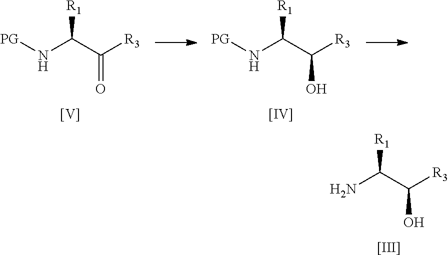

wherein:
R¹ is alkyl or haloalkyl;
R³ is alkyl, haloalkyl or aryl; and
PG is a protecting group;
said process comprising:
(a) treating a compound of formula [V] with (S)-2-Me-CBS-oxazoborolidine and borane-N,N-diethylaniline complex in a solvent comprising THF to form a compound of formula [IV]; and
(b) removing the protecting group PG from said compound [IV] to give a compound of formula [III],
   wherein PG is a protecting group, preferably Boc, R₁ is alkyl or haloalkyl, and R₃ is H, alkyl, haloalkyl or aryl.

In another preferred embodiment, the compound of formula [III] is a compound of formula [IIIa], and is prepared by the steps of:

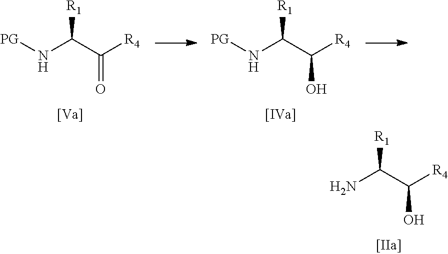

wherein:
R¹ is alkyl or haloalkyl, more preferably alkyl;
R⁴ is alkyl, haloalkyl or aryl, more preferably alkyl; and
PG is a protecting group, preferably BOC;
said process comprising:
(a) treating a compound of formula [Va] with (S)-2-Me-CBS-oxazoborolidine and borane-N,N-diethylaniline complex in a solvent comprising THF to form a compound of formula [IVa]; and
(b) removing the protecting group PG from said compound [IVa] to give a compound of formula [IIIa].

Advantageously, these conditions lead to a highly diastereoselective reduction, imparting a very high diastereomeric excess (ca. 99%) in the resulting intermediate.

In one preferred embodiment, step (b) comprises treating said compound [IV] with gaseous HCl in methanol, concentrating in vacuo, dissolving in ethyl acetate and then sparging with NH₃.

In one highly preferred embodiment, the compound of formula [III] is a compound of formula [3], which is prepared by the steps of:

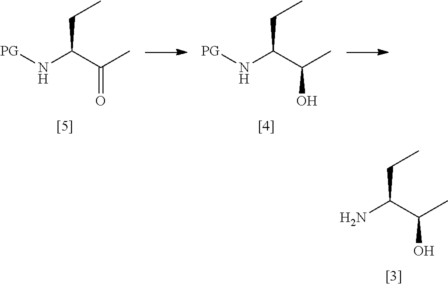

(a) treating a compound of formula [5] with (S)-2-Me-CBS-oxazoborolidine and borane-N,N-diethylaniline complex in a solvent comprising THF to form a compound of formula [4]; and
(b) removing the protecting group PG from said compound of formula [4] to give a compound of formula [3].

Suitable amine protecting groups will be familiar to the skilled person; see for example, Protective Groups in Organic Synthesis by Theodora W. Greene and Peter G. M. Wuts. Preferably, the protecting group PG is a t-butyloxycarbonyl (Boc) group.

Further details of the synthetic process according to the invention are described below, with reference to the reaction scheme set out in Scheme 1:

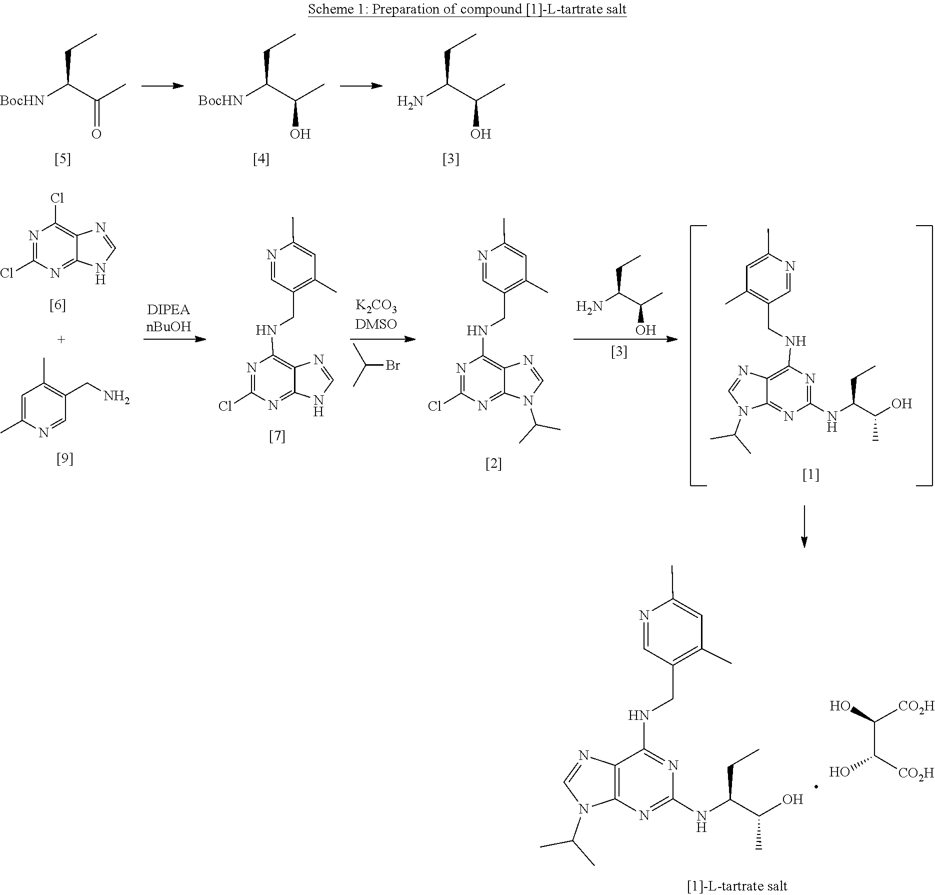

A second aspect of the invention relates to a process for preparing a compound of formula [I], or a pharmaceutically acceptable salt thereof,

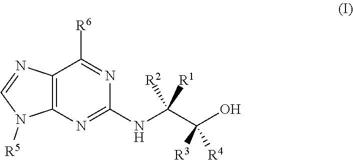

wherein R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and

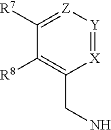

where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of:

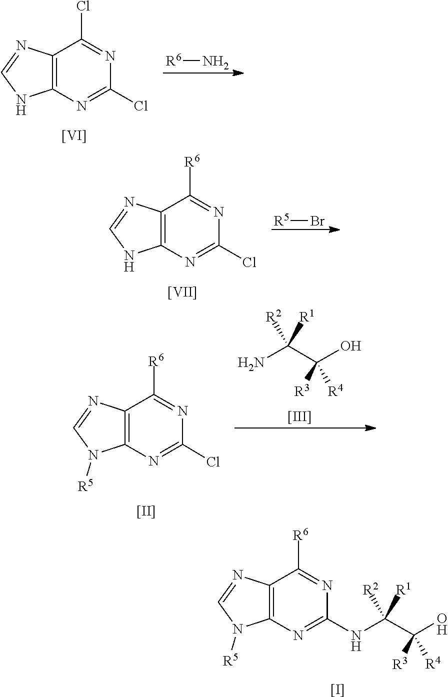

(a) treating a compound of formula [VI] with R⁶—NH₂ or a salt thereof to form a compound of formula [VII];
(b) treating said compound of formula [VII] with R⁵Br to form a compound of formula [II];
(c) forming a reaction mixture comprising a compound of formula [II], and a compound of formula [III] and heating said reaction mixture to a temperature of at least about 130° C. to form a compound of formula [I];
(d) isolating said compound of formula [I] from the mixture and optionally recovering unreacted compound of formula [III]; and
(e) optionally converting said compound of formula [I] into salt form.

In one preferred embodiment of the invention, the process comprises the steps of:
(a) treating a compound of formula [VI] with R⁶—NH₂ or a salt thereof to form a compound of formula [VII];
(b) treating said compound of formula [VII] with R⁵Br to form a compound of formula [II];
(c) forming a reaction mixture comprising a compound of formula [II], and a compound of formula [III] and heating said reaction mixture to a temperature of at least about 130° C. to form a compound of formula [I];
(d) separating said compound of formula [I] from unreacted compound of formula [III]; and
(e) optionally converting said compound of formula [I] into salt form.

A third aspect of the invention relates to a process of preparing a compound of formula [1], or a pharmaceutically acceptable salt thereof, said process comprising the steps of:

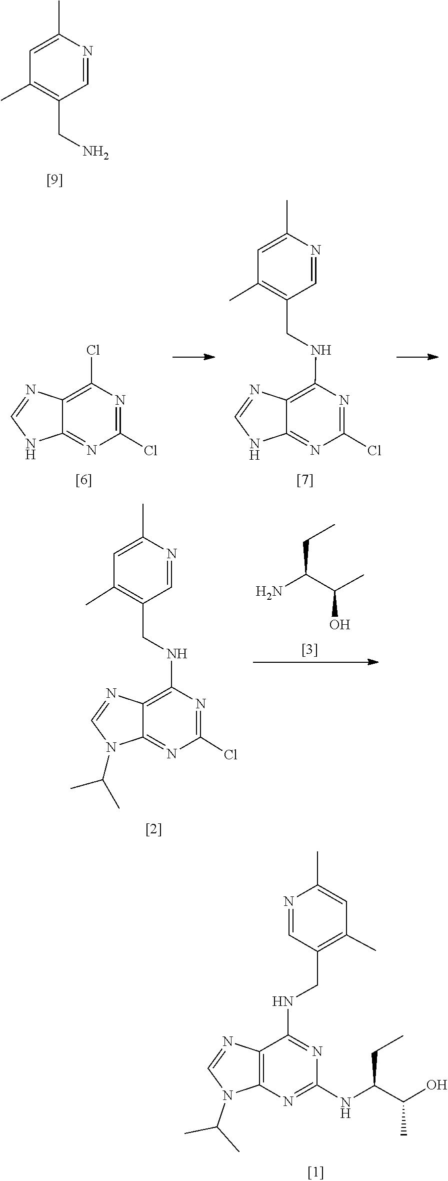

(a) treating a compound of formula [6] with a compound of formula [9], or a salt thereof, to form a compound of formula [7];
(b) treating said compound of formula [7] with isopropyl bromide to form a compound of formula [2];
(c) forming a reaction mixture comprising a compound of formula [2], and compound of formula [3] and heating said reaction mixture to a temperature of at least about 130° C. to form a compound of formula [1];
(d) isolating said compound of formula [1] from the mixture and optionally recovering unreacted compound of formula [3]; and
(e) optionally converting said compound of formula [1] into salt form.

In one preferred embodiment of the invention, the process comprises the steps of:
(a) treating a compound of formula [6] with a compound of formula [9], or a salt thereof, to form a compound of formula [7];
(b) treating said compound of formula [7] with isopropyl bromide to form a compound of formula [2];
(c) forming a reaction mixture comprising a compound of formula [2], and compound of formula [3] and heating said reaction mixture to a temperature of at least about 130° C. to form a compound of formula [1];
(d) separating said compound of formula [1] from unreacted compound of formula [3]; and
(e) converting said compound of formula [1] into salt form.

A fourth aspect of the invention relates to a compound of formula [2]:

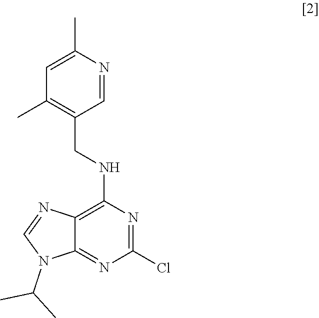 [SMILES: Cc1cc(C)c(CNc2nc(Cl)nc3c2ncn3C(C)C)cn1]

Compound [2] is a useful intermediate in the synthesis of compound [1]. Further aspects of the invention therefore relate to the use of compound [2] as an intermediate in the synthesis of compound [1] as described herein.

Preferred embodiments as described above for the first aspect apply equally to the second, third and fourth aspects.

The present invention is further described with reference to the following figures, wherein:

FIG. 1 shows the reaction scheme for preparing compound [1] as disclosed in WO 2008/122767.

FIG. 2 shows the reaction scheme for preparing compound [1]-L-tartrate as disclosed in WO 2011/089401.

FIG. 3 shows the reaction scheme for preparing compound [1]-L-tartrate as disclosed in WO 2018/138500.

The present invention is further described with reference to the following non-limiting Examples.

### EXAMPLES

### Abbreviations

THF tetrahydrofuran
EtOAc ethyl acetate
PMA phosphomolybdic acid
MeOH methanol
DCM dichloromethane
TBME (MTBE) tertiary butyl methyl ether
DCM dichloromethane

### DIEA N,N-diisopropylethylamine

DMSO dimethyl sulfoxide
¹H NMR: ¹H NMR spectra were collected using a JEOL ECX 400 MHz spectrometer equipped with an auto-sampler. The samples were dissolved in D₆-DMSO for analysis and the spectrum was acquired at ambient temperature using a standard proton experiment acquiring 16 scans using Delta NMR Processing and Control Software version 4.3. The data were then processed using ACD labs 1D NMR processor version 12.0.
DSC: DSC data were collected on a PerkinElmer Pyris 6000 DSC equipped with a 45 position sample holder. The instrument was verified for energy and temperature calibration using certified indium. A predefined amount of the sample, 0.5-3.0 mg, was placed in a pin holed aluminium pan and heated at 20° C.·min⁻¹ from 30 to 350° C., or varied as experimentation dictated. A purge of dry nitrogen at 20 ml·min⁻¹ was maintained over the sample. The instrument control, data acquisition and analysis were performed with Pyris Software v11.1.1 Revision H.
XRPD: X-Ray Powder Diffraction patterns were collected on a PANalytical diffractometer using Cu Kα radiation (45 kV, 40 mA), 6-6 goniometer, focusing mirror, divergence slit (½″), soller slits at both incident and divergent beam (4 mm) and a PIXcel detector. The software used for data collection was X′Pert Data Collector, version 2.2f and the data was presented using X′Pert Data Viewer, version 1.2d. XRPD patterns were acquired under ambient conditions via a transmission foil sample stage (polyimide-Kapton, 12.7 μm thickness film) under ambient conditions using a PANalytical X′Pert PRO. The data collection range was 2.994-35° 2θ with a continuous scan speed of 0.202004° s⁻¹.

### HPLC: Method A

### Sample Solution Preparation:

Accurately weigh 50 mg of sample into a 100 ml volumetric flask. Add 50 ml of Methanol to the flask, dissolve via sonication if necessary, dilute to volume with Purified Water and mix the resulting solution thoroughly.
Column: 150×4.6 mm Luna C18 (2), 5 μm particle size, (ex-Phenomenex; #OOF-4252-EO)
Mobile Phase: A—0.01 M Ammonium Acetate Buffer (pH 8.0)
   B—Acetonitrile
Flow Rate: 1.0 ml·min⁻¹
Injection Volume: 5 μl
Detection: UV@ 254 nm
Column Temp: 30° C.
Post Run: 5 minutes

### HPLC: Method B

### Sample Solution Preparation:

Accurately weigh 50 mg of sample into a 100 ml volumetric flask. Add 50 ml of acetonitrile to the flask, dissolve via sonication if necessary, dilute to volume with Purified Water and mix the resulting solution thoroughly.
Column: 150×4.6 mm XBridge Phenyl, 3.5 μm particle size, (ex-Waters; #186003335)
Mobile Phase: A—Purified Water:Trifluoroacetic acid (100:0.1)
   B—Acetonitrile:Trifluoroacetic acid (100:0.1)
Flow Rate: 1.0 ml·min⁻¹
Injection Volume: 5 μl
Detection: UV@ 268 nm
Column Temp: 30° C.
Post Run: 5 minutes

### Chiral HPLC

Column: 250×4.6 mm Chiralpak AD-H, 5 μm particle size, (ex-Daicel Chemical Industries, Ltd; #DAIC 19325)
Mobile Phase: Ethanol:Hexane (50:50)
Flow Rate: 1.0 ml·min⁻¹
Injection Volume: 20 μl
Detection: UV@ 268 nm
Column Temp: 40° C.
Run Time: 20 minutes

### HRGC:

### Sample Solution Preparation

Accurately weigh 50 mg of sample into a 10 ml volumetric flask. Dissolve in 5 ml of dichloromethane, using sonication if required, dilute to volume with dichloromethane and mix the resulting solution thoroughly.
Column: DB-1 30m×0.32 mm; 1.0 μm film thickness (ex-J&W Scientific #123-1033)
Oven Program: 40° C. (Hold 5 mins) then 10° C.·min⁻¹ to 300° C. (Hold 10 mins)
Injector Temperature: 200° C., split
Column Temperature: 250° C., F.I.D.
Head Pressure: 12 psi, constant pressure
Carrier Gas: Nitrogen
Split Ratio: 50:1
Injection Volume: 2 μl
Liner: SGE Focusliner with glass wool insert

### Enantiomeric Excess by HRGC:

### Standard Solution Preparation

Accurately weigh 10 mg of each enantiomer [(2R,3S); (2S,3R); (2R,3R); (2S,3S)] into a suitable container. Dissolve in about 1 ml of HPLC grade dichloromethane, sonicating if necessary. Add 500 μl of trifluoroacetic anhydride and 500 μl of trifluoroacetic acid and allow to derivatise for 15-30 minutes at room temperature. Inject this solution

### Sample Solution Preparation

Accurately weigh, in duplicate, 10 mg of sample into a suitable container. Dissolve in about 1 ml of HPLC grade dichloromethane, sonicating if necessary. Add 500 μl of trifluoroacetic anhydride and 500 μl of trifluoroacetic acid and allow to derivatise for 15-30 minutes at room temperature. Inject this solution.
Column: Gamma-TA Cyciodextrin 30 m×0.32 mm;
   0.125 μm film (ex-Astec; Cat no. 73033)
Oven Program: 80° C. (Hold 10 mins) then 2° C.·min⁻¹ to 90° C.
   (Hold 20 mins) then 10° C.·min⁻¹ to 80° C.
Injector Temperature: 200° C., split
Column Temperature: 250° C., F.I.D.
Head Pressure: 20 psi, constant pressure
Carrier Gas: Nitrogen
Spilt Ratio: 50:1
Injection Volume: 1 μl
Liner: SGE Focusliner with glass wool insert

$$\% enantiomeric excess = \frac{Peak area of [ ( 2 R , 3 S ) - ( 2 S , 3 R ) ]}{Peak area of [ ( 2 R , 3 S ) + ( 2 S , 3 R ) ]} \times 100 \%$$

$$\% diastereomeric excess = \frac{Peak area of [ ( 2 R , 3 S ) - ( 2 S , 3 S ) ]}{Peak area of [ ( 2 R , 3 S ) + ( 2 S , 3 S ) ]} \times 100 \%$$

### Example A

### (2R,3S)-3-aminopentan-2-ol synthesis

### Preparation of Compound [4]

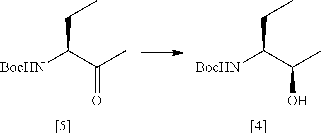

(S)-2-Methyl-CBS-oxaborolidine (1M solution in toluene, 59.6 mL, 0.06 mol) was diluted with THF (171 mL) in a dry, nitrogen purged vessel. Borane N,N-diethylaniline complex (102 mL, 0.57 mol) was added dropwise at room temperature and the solution was allowed to stir for 15 minutes. Compound [5] (115.0 g, 0.57 mol) was dissolved in THF (345 mL) and added dropwise over 4.5 h. After the addition was complete the reaction was allowed to stir overnight at room temperature under a nitrogen atmosphere. Thin layer chromatography (20% EtOAc in heptane, visualised by PMA) indicated the complete consumption of starting material. The reaction was carefully quenched via dropwise addition of methanol (174 mL) over 1 h. The temperature was maintained at <20° C. throughout the quench. The solution was concentrated in vacuo before additional methanol (174 mL) was added. The solution was concentrated under reduced pressure to afford a white waxy solid. The crude product was recrystallised from heptane (202 mL). The recrystallised product was filtered and rinsed with heptane (2×156 mL) to yield a white solid. This was dried in a vacuum oven at 40° C. overnight to give compound [4] as a white solid (99.2 g, 85%). Analysis was by HRGC and chiral HRGC as described above.

### Example B

### Deprotection of Compound [4]

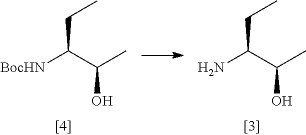

MeOH (645 mL) was gassed with HCl for 1 hour at <20° C. in a 20 L flask under N₂. The solution was 3.85M by titration. The flask was cooled to <15° C. Compound [4] (101.1 g, 0.50 mol) was charged portion-wise at <15° C. The solution was stirred overnight. The reaction was complete by TLC (5% MeOH/DCM, visualised with PMA). The solution was concentrated in vacuo at 35-40° C. The oil was azeotroped with EtOAc (4×75 mL) and triturated to give a white solid. The solid was taken up in EtOAc (588 mL). The reaction mixture was cooled to 0-5° C. and sparged with NH₃ (g) for 1 hour at 0-5° C. under N₂. At the end of the addition the pH was 8. The mixture was filtered and the filter cake was washed with EtOAc (147 mL). The filtrate was concentrated in vacuo at 35-40° C. to give the desired product [3] as a light yellow oil (50.7 g). ¹H NMR confirmed the identity of the product and indicated ˜4% residual EtOAc to be present giving an active yield of 48.7 g, 95%.

### Example C

### Synthesis of Compound [7]

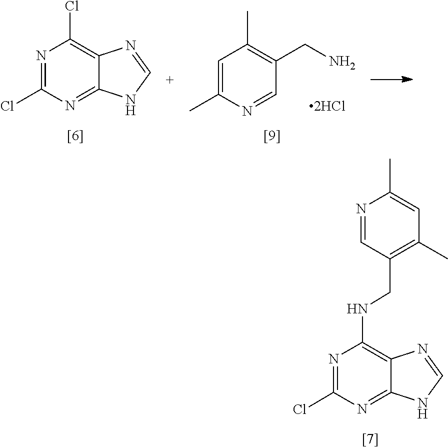

Compound [6] (90 g, 0.48 mol) and compound [9] (100 g, 0.48 mol) are charged to a vessel along with n-butanol (1.98 L) and DIEA (295 mL, 1.68 mol). The reaction mixture was heated to 110° C. under N₂ for 22 hours. The reaction was monitored by ¹H NMR to confirm that the starting material had been consumed. The reaction was cooled to room temperature and stirred at 15-25° C. for 30 min. The reaction mixture was filtered and washed 3× with TBME (3×125 mL). The product was dried in vacuo at 40° C. for 68 hours to yield compound [7] (124 g, 89%). ¹H NMR confirmed the identity of the product and HPLC (Method A) indicated a purity of 99.2%.

### Example D

### Synthesis of Compound [2]

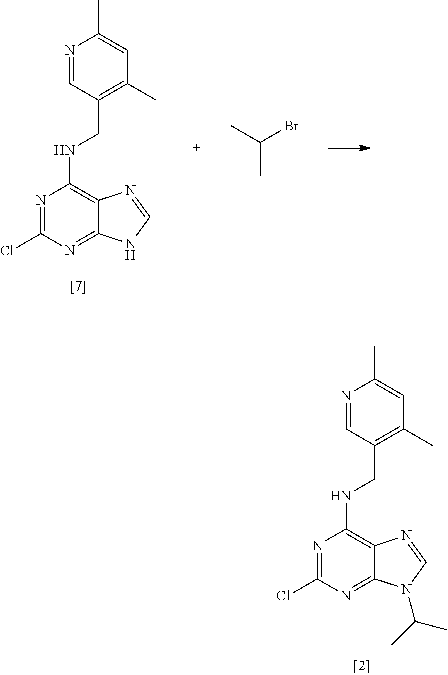

Compound [7] (120 g, 0.416 mol), potassium carbonate (115 g, 0.832 mol) and DMSO (1.2 L) are charged to a vessel. 2-bromopropane (117 ml, 1.248 mol) is added portion wise over 2 minutes. The reaction mixture is heated to 60° C. and stirred for 50 minutes. The reaction was monitored by HPLC (Method A) to confirm that the starting material had been consumed. The reaction was cooled and water (1.2 L) was added and mixture was extracted 3× with ethyl acetate (3×0.9 L). The combined organic phase was washed 6× with water (6×1.1 L) and dried with magnesium sulphate. The magnesium sulphate was filtered off and the organic phase was evaporated to dryness on a rotary evaporator. The crude product was purified through a plug of silica using 15% methanol in ethyl acetate as eluent. The fractions containing the product were evaporated to dryness on a rotary evaporator to yield compound [2] (130 g, 93%). ¹H NMR confirmed the identity of the product and HPLC (Method A) indicated a purity of 99.2%.

In an alternative embodiment, compound [2] is prepared by treating compound [6] with 2-bromopropane to form compound [8] using similar conditions and reagents to those described above. Compound [8] is then reacted with compound [9] in the presence of n-butanol and DIEA using similar conditions to those described above to form compound [2].

### Example E

### Synthesis of Compound [1] (Crude)

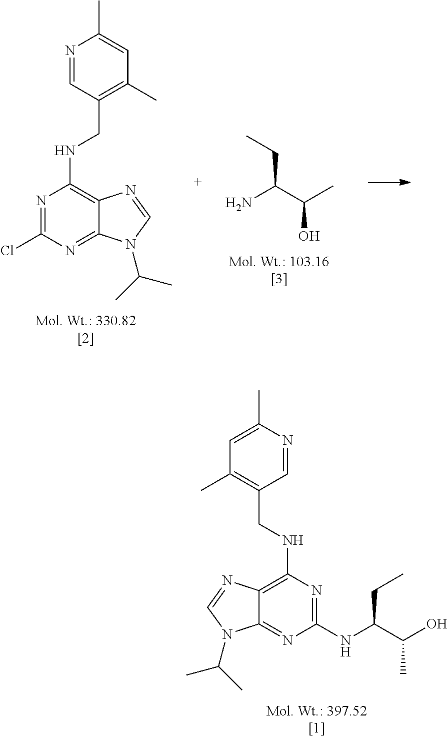

Compound [2] (25 g, 0.0768 mol) and compound [3] (39.6 g, 0.384 mol) were charged to a vessel and heated to 170° C. (heating block temperature) under nitrogen for 48 hours. The reaction was monitored by HPLC (Method B) for the disappearance of Compound [2].

### Recovery of Compound [3] by Distillation

The reaction was cooled to 60° C. and PEG400 (80 mL) and 8M sodium hydroxide solution (9.4 mL) were added. The reaction mixture was fractionally distilled in vacuo at 30 to 50 mBar and at temperatures from 80 to 170° C. A fraction was collected at a head temperature of 88° C. which contained Compound [3] (25 g, 71% recovery). The fraction was analysed by ¹H NMR and indicated a purity of >95%. The fraction was also analysed for what content by Karl Fischer titration which indicated that the fractioned contained 12% water.

“Butyl acetate (330 mL) and brine (650 mL) were added to the distillation pot residue and stirred. The organic phase was separated and the aqueous phase was re-extracted with “Butyl acetate (170 mL). The combined organic layers were washed 4× with water (4×250 mL) and dried over magnesium sulphate, filtered and the solvent was removed in vacuo to give the crude Compound [1] (29 g, 86% yield). The product was analysed by ¹H NMR which indicated that it contained 11% “Butyl acetate) and also by HPLC (Method B) which indicated a purity of 95.5%.

### Example F

### Crystallisation of Crude Compound [1]

### Example F.1

Crude Compound [1] (28 g) was dissolved in “Butyl acetate (130 ml) and heated to 70° C. and then cooled to 66° C. and seed crystals of compound [1] were added and stirred for 1h. The seed crystals of compound [1] are designated as Form A and are as described in WO 2011/089401 (Cyclacel Limited; see in particular, Example 1—reproduced below), the contents of which are hereby incorporated by reference. The mixture was cooled to 25° C. over 14 hours whereupon a slurry formed. The slurry was stirred for 6 hours and then heptane (60 ml) was added over 1 hour 20 min and then stirred for 1 hour. The heptane functions as an antisolvent to increase the yield. The slurry was then cooled to 0° C. over 1 hour and stirred for 1 hour. The product was filtered in vacuo and washed 2× with 2:1 “Butyl acetate:heptane (2×20 ml) and then dried in vacuo at 45° C. for 18 hours to give Compound [1] (20.8 g, 69% overall yield (83% (crystallisation yield). The product was analysed by HPLC (Method B) which indicated a purity of 98.3% and also by XRPD which indicated that the product was crystals of Form A (in accordance with Example 1 of WO 2011/089401). XRPD peaks for crystalline free base (Form A) of Compound [1] are shown in Table 1.

### Example F.2

### Preparation of Seed Crystals of Compound [1] (Form A) as Described in Example 1 of WO 2011/089401

Compound [1] was crystallised from MTBE by the following method. MTBE (2 vol) was added to compound [1] and heated to reflux. The mixture was held at reflux for 30-60 minutes before the temperature was reduced to 50° C. (held for 2 hours). The suspension was allowed to cool slowly to room temperature before being filtered and rinsed with MTBE (3×1 vol). The solids were dried in vacuum oven at 40° C. for 8 hours to afford the desired crystalline free base (mass recovery 84.5%, LC purity 97.4%).

### Example F.3

### Alternative Crystallisation Conditions for Crude Compound [1] Isopropyl Acetate, 5 Volumes, 85° C., 6 g Scale

Crude Compound [1] (6.09 g) was suspended in isopropyl acetate (30 ml, 5 volumes) in a suitable glass vessel under N₂ fitted with reflux condenser and stirrer bead agitation at 420 RPM. The beige suspension was heated to 85° C. (target 80-85° C.) upon which full dissolution to a dark brown solution was achieved after ca. 15 minutes at temperature.

The hot mixture was clarified through a heated large nylon syringe filter (0.44 μm) into a Mya4 100 ml process vessel with overhead U-shape agitation at 200 RPM pre-heated to 95° C. (target internal temperature=80-85° C.). The dark brown solution was cooled to 74.5° C. over 15 minutes and seeded with crystals of compound [1] (Form A) (ca. 6 mg 0.1% wt). Cooling was continued to 70.1° C. and seeding repeated, which was observed to maintain in solution after 15 minutes at temperature. A T=0 solubility measurement was taken (189.83 mg/ml) before the mixture was then cooled to 0° C. at a rate of 5° C./hour (14 hours, 840 minutes). Noticeable amounts of off-white precipitate were observed ca. 61-62° C. Following cooling, the mixture was held at 0° C. for ca. 3 hours. The solids were isolated via filtration, with no evidence of fouling of the vessel or agitator. The vessel and filter cake were washed with the isolated filtrates and air dried for 15 minutes before collection of the off-white solids and dark brown liquors. The solids were collected and dried in vacuo at 45° C. for ca. 4 hours to yield the title compound. 4.88 g of crystalline Compound [1] was successfully isolated in 80.1% yield. The maximum theoretical recovery based upon measured solubility pre-isolation at 0° C. was 94.2%.

### Example F.4

### Ethyl Acetate, 5 Volumes, 75° C., 6 g Scale

Crude Compound [1] (6.00 g) was suspended ethyl acetate (30 ml, 5 volumes) in a suitable glass vessel under N₂ fitted with reflux condenser and stirrer bead agitation at 420 RPM. The beige suspension was heated to 75° C. (target 70-75° C.) upon which full dissolution to a dark brown solution was achieved during heating at ca. 68° C. The hot mixture was clarified through a heated large nylon syringe filter (0.44 μm) into a Mya4 100 ml process vessel with overhead U-shape agitation at 200 RPM pre-heated to 85° C. (target internal temperature=70-75° C.). The dark brown solution was cooled to 65.2° C. over 15 minutes (target 62.5-67.5° C.) and seeded with crystals of compound [1] (Form A) (ca. 6 mg 0.1% wt). Cooling was continued to 59.8° C. (target 57.5-62.5° C.) and seeding repeated, which was observed to maintain in solution after ca. 20 minutes at temperature. A T=0 solubility measurement was taken (184.35 mg/ml) before the mixture was then cooled to 0° C. at a rate of 5° C./hour (12 hours, 720 minutes). Noticeable amounts of an off-white precipitate were observed at ca. 52-53° C. Following cooling, the mixture was held at 0° C. for ca. 5 hours before solubility was measured indicating 90.06% development (18.84 mg/ml). The solids were isolated via filtration. The vessel and filter cake were washed with the isolated filtrates and air dried for 12 minutes before collection of the off-white solids and dark brown liquors. The solids were collected and dried in vacuo at 45° C. for ca. 4 hours to yield the title compound. 4.59 g of crystalline Compound [1] was successfully isolated in 76.4% yield. The maximum theoretical recovery based upon measured solubility pre-isolation at 0° C. was 90.6%.

### Example F.5

n-Butyl Acetate, 5 Volumes, 85° C., 6 g Scale

Crude Compound [1] (5.96 g) was suspended n-Butyl acetate (30 ml, 5 volumes) in a suitable glass vessel under N₂ fitted with reflux condenser and stirrer bead agitation at 420 RPM. The beige suspension was heated to 85° C. (target 80-85° C.) upon which full dissolution to a dark brown solution was achieved during heating at ca. 82° C. The hot mixture was clarified through a heated large nylon syringe filter (0.44 μm) into a Mya4 100 ml process vessel with overhead U-shape agitation at 200 RPM pre-heated to 95° C. (target internal temperature=80-85° C.). The dark brown solution was cooled to 74.6° C. over 15 minutes (target 72.5-77.5° C.) and seeded with crystals of compound [1] (Form A) (ca. 6 mg 0.1% wt). Cooling was continued to 69.5° C. (target 67.5-72.5° C.) and seeding repeated, which was observed to maintain in solution after 45 minutes at temperature. A T=0 solubility measurement was taken (175.84 mg/ml) before the mixture was then cooled to 0° C. at a rate of 5° C./hour (14 hours, 840 minutes). Development of a small amount of large particulates were observed at ca. 60-62° C. Following cooling, the mixture was held at 0° C. for ca. 3 hours before solubility was measured indicating 91.7% development (16.61 mg/ml). The solids were isolated via filtration. The vessel and filter cake were washed with the isolated filtrates and air dried for 10 minutes before collection of the grey solids and dark brown liquors. The solids were collected and dried in vacuo at 45° C. for ca. 4 hours to yield the title compound. 4.64 g of crystalline Compound [1] was successfully isolated in 77.9% yield. The maximum theoretical recovery based upon measured solubility pre-isolation at 0° C. was 91.7%.

### Example F.6

Crystallisation of Crude Compound [1] in n-Butyl Acetate (5 Volumes, 85° C.) Using Heptane Anti-Solvent Addition

The crystallisation procedure outlined in Example F.5 was followed with the following modifications:
Heptane (2.5 volume) anti-solvent addition performed at 25° C. at a rate of 1 volume/hour following controlled cool at 5° C./hour;
Heptane (2.5 volume) anti-solvent addition performed at 70° C. following seeding at a rate of 1 volume/hour, followed by controlled 5° C./hour cool to 25° C.

As the final composition of the crystallisation contains a mixed solvent system, the impact of a standard 2×2 volume vessel and filter cake rinse using the final solvent composition of n-Butyl acetate/Heptane [2:1] was assessed.

n-Butyl Acetate, 5 Volumes, 85° C., 6 g Scale Crystallisation with Heptane (2.5 Volume) Anti-Solvent Addition Performed at 25° C. at a Rate of 1 Volume/Hour Following Controlled Cool at 5° C./Hour

4.73 g of crystalline Compound [1] was successfully isolated in 78.5% yield. The maximum theoretical recovery based upon measured solubility pre-isolation at 25° C. was 92.4%.

n-Butyl Acetate, 5 Volumes, 85° C., 6 g Scale Crystallisation with Heptane (2.5 Volume) Anti-Solvent Addition Performed at 70° C. at a Rate of 1 Volume/Hour Followed by Controlled Cool at 5° C./Hour to 25° C.

4.73 g of crystalline Compound [1] was successfully isolated in 76.0% yield. The maximum theoretical recovery based upon measured solubility pre-isolation at 25° C. was 92.4%.

### Example G

### Synthesis of Compound [1]-L-Tartrate Salt

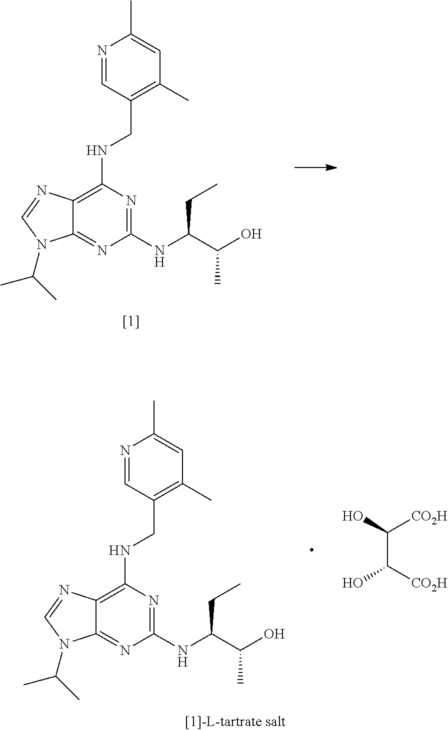 [SMILES: CC[C@H](Nc1nc(NCc2cnc(C)cc2C)c2ncn(C(C)C)c2n1)[C@@H](C)O ; CC[C@H](Nc1nc(NCc2cnc(C)cc2C)c2ncn(C(C)C)c2n1)[C@@H](C)O ; O=C(O)[C@H](O)[C@@H](O)C(=O)O]

Crystalline compound [1] free base (29.9 g, 75.22 mmol) was dissolved in ethanol (420 mL) and the resulting solution heated at reflux. A solution of L-tartaric acid (11.29 g, 75.22 mmol) in water (12 mL)/ethanol (30 mL) was added dropwise maintaining the batch temperature at 75-78° C. The solution was polish filtered (cooled to 57° C. during filtration with no evidence of crystallisation). The filtered solution was warmed to 60-65° C. and seeded with compound [1]-L-tartrate salt form 11 (Form E) (0.003 g) prepared in accordance with Example 5 of WO 2011/089401 (reproduced below; Cyclacel Limited). The mixture was stirred at 60-65° C. for 1 hour during which time crystallisation initiated. The suspension was then cooled to 15-20° C. at 10° C./h. After stirring at 15-20° C. for 1 hour, the solid was filtered, washed with ethanol (3×60 mL) and pulled dry. Further drying in a vacuum oven yielded [1]-L-tartrate salt as a white solid (36.0 g, 87% from free base). ¹H NMR confirmed the identity of the product and HPLC (Method B) indicated a purity of 98.80%. The product was also analysed by chiral HPLC. DSC analysis (peak 182.73° C., onset 179.61° C.) and XRPD confirmed form E in accordance with WO 2011/089401. XRPD peaks for L-tartrate (Form E) of Compound [1] are shown in Table 2.

### Preparation of Seed Crystals of L-Tartrate Salt (Form E) of Compound [1] According to Examples 4 and 5 of WO 2011/089401

### Example 4: Preparation of L-Tartrate Salt (Form D) of Compound [1]

Compound [1] (500 mg, 1.26 mmol, 1 equiv.), L-tartaric acid (193 mg, 1.28 mmol, 1.02 equiv) and ethyl acetate (5 ml, 10 vol) were charged to a flask and stirred under ambient conditions for 2 hours, precipitation occurred inside 1 hour. The white precipitate was isolated by vacuum filtration, washed with EtOAc (3×0.5 ml, 2×1 ml) and dried in a vacuum oven at 40° C. for 16 hours to yield the L-tartrate salt as a white solid (565 mg, 82% yield; Form D).

### Example 5.1

A suspension of Form D L-tartrate salt of compound [1] as prepared in Example 4 of WO 2011/089401 above (1.0 g) in ethanol (12 ml) was heated at reflux. Acetonitrile (3 ml) was added portion wise over 30 minutes. After this addition, a solution was not obtained. Further portions of ethanol (4.5 ml) and acetonitrile (1 ml) were added until a solution was obtained. The solution was polish filtered (hot) then cooled to room temperature at a rate of 10° C./hour (crystallisation initiated at ˜65° C.). After stirring at room temperature overnight, the resulting solid was filtered, washed with cold ethanol (5 ml) and pulled dry. Further drying in a vacuum oven at 50° C. yielded the desired product as a white crystalline solid (0.725 g, 73%). ¹H NMR analysis confirmed a 1:1 salt and XRPD confirmed Form E.

### Example 5.2

A suspension of Form D L-tartrate salt of compound [1] as prepared in Example 4 of WO 2011/089401 above (10.2 g) in ethanol (120 ml) was heated to 65° C. Acetonitrile (20 ml) was added and the suspension heated at reflux for 10 minutes after which time a solution was obtained. The solution was cooled to room temperature over 2-3 hours with crystallisation initiating at −50° C. The resulting suspension was stirred at room temperature overnight. The resulting solid was filtered, washed with ethanol (10 ml) and pulled dry. Further drying in a vacuum oven at 50° C. yielded the desired product as a white crystalline solid (8.76 g, 88%). ¹H NMR analysis confirmed a 1:1 salt and XRPD confirmed Form E.

### Example 5.3—Slurry Conversion

Form E of the L-Tartrate salt of compound [1] was also prepared by slurry conversion from four different solvents (ethyl acetate, IPA, IMS or acetonitrile). A 1:1 mixture of Form by weight of D:Form E L-Tartrate salt (200 mg total) was heated at 45° C. over 48 hours in 2 ml of solvent prior to filtration and analysis. Form E was produced in each slurry (purity 98%).

### Example 5.4—Seeding

A suspension of Form D L-tartrate salt compound [1] as prepared in Example 4 of WO 2011/089401 above (10.2 g) in ethanol (120 ml) was heated to 65° C. Acetonitrile (20 ml) was added and the suspension heated at reflux for 10 minutes. The mixture was polish filtered through HPLC filter frits. No precipitation was observed in process. The material was then cooled from reflux and seeded at 70° C. with Form E L-tartrate salt (as prepared above), cooling at a rate of 10° C. every 1.5 hours. The first seed dissolved completely and seeding was repeated at 60° C. The seed remained and the solution changed to show a very faint opaque phase. Crystallisation began at approximately 50° C. An isolated yield of 80% was obtained.

### Example 5.5—Formation from Free Base of Compound [1]

Compound [1] free base Form A (0.2 g) was dissolved in ethanol (9 vol, 1.8 mL) and heated at reflux. A solution of tartaric acid (1 eq, 0.076 g) in water (1.7 vol, 0.34 mL)/ethanol (1 vol, 0.2 mL) was added dropwise maintaining the temperature at reflux. The resulting solution was then polish filtered before cooling to 70° C. A seed of Form E was added giving a cloudy solution. The batch was stirred at 70° C. for 1 hour before cooling to room temperature. After stirring at room temperature for 2 hours, the solid was filtered, washed with ethanol (2×0.5 mL) and pulled dry. Further drying in a vacuum oven at 50° C. yielded Compound [1]-L-tartrate salt Form E as a white solid (0.2 g, 72%). ¹H NMR confirmed a 1:1 salt and HPLC indicated a purity of 97.97%. XRPD and DSC confirmed Form E.

### Example H

Preparation of Compound [1] with Direct Isolation as the L-Tartrate Salt

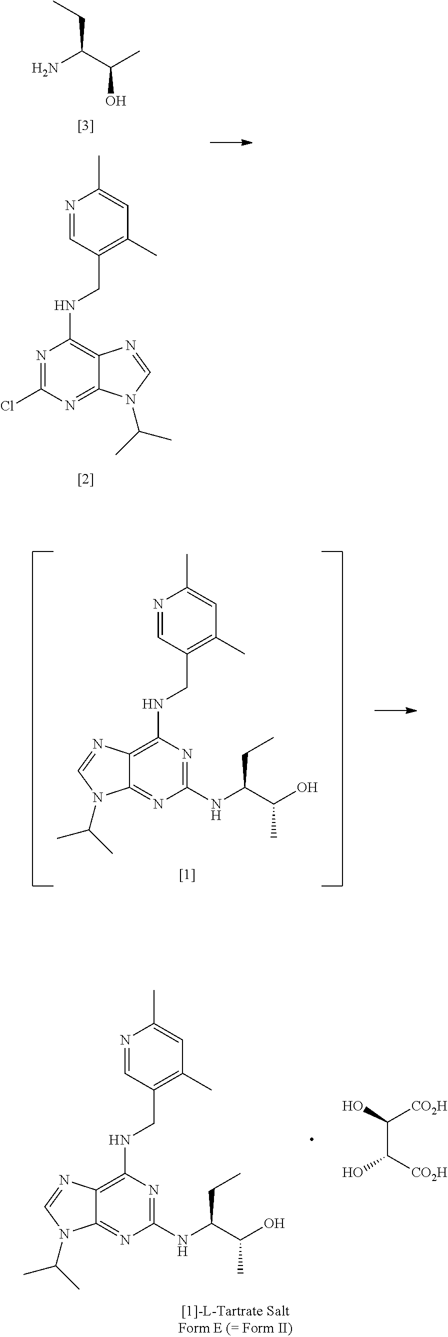

Compound [2] (25 g, 0.0768 mol) and compound [3] (39.6 g, 0.384 mol, 5 mol eq) were charged to a vessel and heated to 170° C. (heating block temperature) under nitrogen for 48 hours. The reaction was monitored by HPLC (Method B) for the disappearance of Compound [2].

The reaction mixture was cooled to 60° C. The remaining compound [3] content was determined by ¹H NMR and 1 mol eq of HCl (as 4M HCl) relative to the amount of remaining compound [3] was charged. Ethyl acetate (10 vol) was charged and stirred to extract Compound [1] into the organic phase. The aqueous phase was separated and re-extracted with a further 10 vol of ethyl acetate. The organic phases were combined and washed with water (10 vol).

The organic phase was concentrated via distillation to approximately 5 volumes. Ethanol (10 vol) was charged and the distillation continued to remove the ethyl acetate. Further portions of ethanol were charged and the distillation continued until the ethyl acetate had been removed.

Sufficient ethanol was charged such that concentration of Compound [1] was 14 volumes. The mixture was heated to 75-78° C. L-tartaric acid (1 mol eq) was dissolved in purified water:ethanol (1:2.5 ratio, 1.4 vol relative to Compound [1]. The L-tartaric solution was added dropwise to the ethanol solution of Compound [1] at 75-78° C. The mixture was cooled to 60-65° C. and seeded with Tartrate Salt (Form E) of Compound [1]. The mixture was stirred at 60-65° C. for 1 hour until the crystallisation initiated. The suspension was cooled to 15-25° C. at 10° C./h. The suspension was stirred for 1 h at 15-25° C. and then filtered in vacuo, washed with ethanol (3×2 vol) and dried in vacuo at 50° C. Expected yield is 65-70% (29.4 g @ 70% yield).

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
XRPD peaks for crystalline free base (Form A) of Compound [1]

| Pos. | Height | FWHM | d-spacing | Rel. Int. | Tip width |
|---|---|---|---|---|---|
| [°2Th.] | [cts] | [°2Th.] | [Å] | [%] | [°2Th.] |
| 7.5313 | 21162.64 | 0.0768 | 11.73852 | 100.00 | 0.0921 |
| 9.6026 | 846.92 | 0.0768 | 9.21066 | 4.00 | 0.0921 |
| 10.2275 | 400.15 | 0.1023 | 8.64925 | 1.89 | 0.1228 |
| 11.2954 | 1863.94 | 0.1023 | 7.83384 | 8.81 | 0.1228 |
| 11.6652 | 300.53 | 0.1023 | 7.58631 | 1.42 | 0.1228 |
| 12.2672 | 3812.02 | 0.1023 | 7.21534 | 18.01 | 0.1228 |
| 12.6242 | 497.83 | 0.1023 | 7.01205 | 2.35 | 0.1228 |
| 13.1780 | 953.85 | 0.1023 | 6.71859 | 4.51 | 0.1228 |
| 14.0653 | 4092.77 | 0.1023 | 6.29672 | 19.34 | 0.1228 |
| 14.8535 | 1458.15 | 0.0768 | 5.96431 | 6.89 | 0.0921 |
| 15.1515 | 343.64 | 0.0768 | 5.84765 | 1.62 | 0.0921 |
| 15.5775 | 2894.50 | 0.1279 | 5.68868 | 13.68 | 0.1535 |
| 16.9914 | 2108.17 | 0.1023 | 5.21838 | 9.96 | 0.1228 |
| 17.6862 | 1501.97 | 0.1279 | 5.01490 | 7.10 | 0.1535 |
| 18.3040 | 644.51 | 0.0591 | 4.84701 | 3.05 | 0.0709 |
| 18.3954 | 1212.49 | 0.0768 | 4.82314 | 5.73 | 0.0921 |
| 18.6301 | 1666.18 | 0.1023 | 4.76289 | 7.87 | 0.1228 |
| 18.9784 | 1639.81 | 0.1279 | 4.67626 | 7.75 | 0.1535 |
| 19.3292 | 475.31 | 0.1023 | 4.59219 | 2.25 | 0.1228 |
| 20.2061 | 1067.39 | 0.1023 | 4.39483 | 5.04 | 0.1228 |

TABLE 2
XRPD peaks for L-tartrate salt (Form E) of Compound [1]

| Pos. | Height | FWHM | d-spacing | Rel. | Tip width |
|---|---|---|---|---|---|
| [°2Th.] | [cts] | [°2Th.] | [Å] | Int. [%] | [°2Th.] |
| 6.6675 | 15483.76 | 0.0768 | 13.25733 | 100.00 | 0.0921 |
| 8.2340 | 241.15 | 0.1023 | 10.73824 | 1.56 | 0.1228 |
| 9.7722 | 479.22 | 0.1023 | 9.05118 | 3.09 | 0.1228 |
| 11.9598 | 926.89 | 0.1023 | 7.40005 | 5.99 | 0.1228 |
| 12.3792 | 494.24 | 0.0768 | 7.15029 | 3.19 | 0.0921 |
| 13.0632 | 4104.92 | 0.0768 | 6.77739 | 26.51 | 0.0921 |
| 13.3777 | 2386.00 | 0.1023 | 6.61876 | 15.41 | 0.1228 |
| 13.9359 | 413.30 | 0.0768 | 6.35490 | 2.67 | 0.0921 |
| 14.9035 | 1349.55 | 0.1023 | 5.94439 | 8.72 | 0.1228 |
| 15.4032 | 975.17 | 0.0768 | 5.75266 | 6.30 | 0.0921 |
| 15.9507 | 949.23 | 0.1023 | 5.55642 | 6.13 | 0.1228 |
| 16.2665 | 488.77 | 0.1023 | 5.44926 | 3.16 | 0.1228 |
| 16.5423 | 792.08 | 0.1023 | 5.35902 | 5.12 | 0.1228 |
| 17.3614 | 2687.54 | 0.1023 | 5.10799 | 17.36 | 0.1228 |
| 17.5690 | 1410.91 | 0.1023 | 5.04809 | 9.11 | 0.1228 |
| 17.8630 | 201.26 | 0.1023 | 4.96566 | 1.30 | 0.1228 |
| 19.6395 | 1756.56 | 0.0768 | 4.52032 | 11.34 | 0.0921 |
| 19.8636 | 777.97 | 0.0768 | 4.46982 | 5.02 | 0.0921 |
| 20.1195 | 549.42 | 0.1023 | 4.41355 | 3.55 | 0.1228 |
| 20.7288 | 1423.91 | 0.1279 | 4.28518 | 9.20 | 0.1535 |
| 21.1373 | 389.18 | 0.1279 | 4.20327 | 2.51 | 0.1535 |
| 21.5804 | 674.89 | 0.1535 | 4.11797 | 4.36 | 0.1842 |
| 22.5683 | 459.02 | 0.1535 | 3.93989 | 2.96 | 0.1842 |
| 22.9541 | 780.05 | 0.1279 | 3.87454 | 5.04 | 0.1535 |
| 23.2869 | 904.34 | 0.1023 | 3.81992 | 5.84 | 0.1228 |
| 23.5693 | 1652.40 | 0.1535 | 3.77478 | 10.67 | 0.1842 |
| 24.0730 | 899.56 | 0.1535 | 3.69692 | 5.81 | 0.1842 |
| 24.6316 | 316.32 | 0.1791 | 3.61434 | 2.04 | 0.2149 |
| 25.2971 | 1357.36 | 0.1535 | 3.52074 | 8.77 | 0.1842 |
| 26.3772 | 346.67 | 0.1023 | 3.37898 | 2.24 | 0.1228 |
| 27.0905 | 141.69 | 0.1023 | 3.29160 | 0.92 | 0.1228 |
| 27.6723 | 474.86 | 0.1023 | 3.22371 | 3.07 | 0.1228 |
| 27.9727 | 708.87 | 0.1535 | 3.18977 | 4.58 | 0.1842 |
| 28.9051 | 262.52 | 0.1535 | 3.08896 | 1.70 | 0.1842 |
| 29.2843 | 136.18 | 0.1535 | 3.04982 | 0.88 | 0.1842 |
| 30.0801 | 73.71 | 0.1535 | 2.97092 | 0.48 | 0.1842 |
| 30.4059 | 137.17 | 0.1279 | 2.93982 | 0.89 | 0.1535 |
| 31.9006 | 27.79 | 0.1535 | 2.80541 | 0.18 | 0.1842 |
| 34.4898 | 70.18 | 0.2047 | 2.60050 | 0.45 | 0.2456 |

1. A process for preparing a compound of formula [I], or a pharmaceutically acceptable salt thereof, 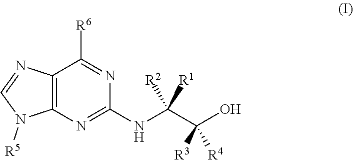
wherein:
R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and 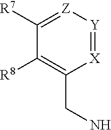
where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of: 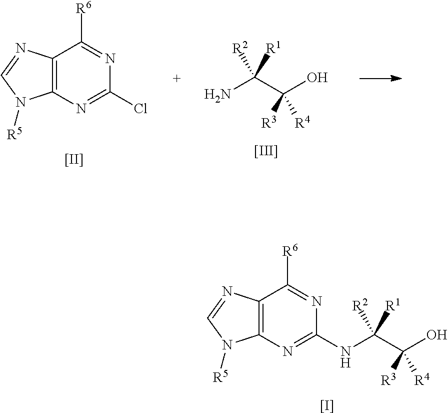
(i) forming a reaction mixture comprising a compound of formula [II], and a compound of formula [III];
(ii) heating said reaction mixture to a temperature of at least about 130° C. to form a compound of formula [I];
(iii) isolating said compound of formula [I] from the mixture and optionally recovering unreacted compound of formula [III]; and
(iv) optionally converting said compound of formula [I] into salt form.

2. A process according to claim 1 wherein the reaction takes place in the absence of a solvent.

3. A process according to any preceding claim wherein the reaction mixture in step (i) is heated to a temperature of from about 135° C. to about 175° C., more preferably from about 150° C. to about 175° C.

4. A process according to any preceding claim wherein the reaction mixture in step (i) is heated for a period of at least 24 hours.

5. A process according to any preceding claim wherein the reaction mixture in step (ii) comprises from about 4 to about 7 mole equivalents of compound [III] relative to compound [II], more preferably about 5 mole equivalents of compound [III] relative to compound [II].

6. A process according to any preceding claim wherein step (iii) comprises extracting the reaction mixture from step (ii) into aqueous HCl and an organic solvent, separating the organic phase and concentrating the filtrate.

7. A process according to any preceding claim which comprises preparing a compound of formula [II] by the steps of: 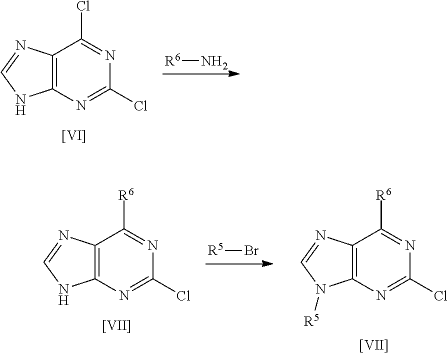
(i) treating a compound of formula [VI] with R⁶—NH₂, or a salt thereof, to form a compound of formula [VII]; and
(ii) treating said compound of formula [VII] with R⁵Br to form a compound of formula [II];
where R⁵ and R⁶ are as defined in claim 1.

8. A process according to any one of claims 1 to 6 which comprises preparing a compound of formula [II] by the steps of: 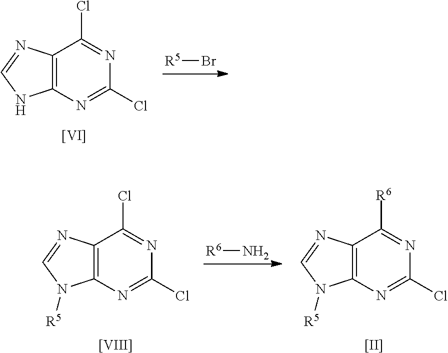
treating a compound of formula [VI] with R⁵Br to form a compound of formula [VIII]; and
(ii) treating said compound of formula [VIII] with R⁶—NH₂, or a salt thereof, to form a compound of formula [II];
where R⁵ and R⁶ are as defined in claim 1.

9. A process according to claim 7 or claim 8 wherein step (ii) of claim 7 or step (i) of claim 8 is carried out in the presence of DMSO and K₂CO₃.

10. A process according to claim 7 or claim 8 wherein step (i) of claim 7 or step (ii) of claim 8 is carried out in the presence of nBuOH and a base, preferably, DIPEA.

11. A process according to any preceding claim wherein step (iii) of claim 1 further comprises the step of crystallizing compound [I], preferably from a mixture of n-butyl acetate and heptane.

12. A process according to any one of claims 1 to 10 which comprises converting said compound of formula [I] into salt form.

13. A process according to any preceding claim which comprises converting said compound of formula [I] into the L-tartrate salt.

14. A process according to claim 13 wherein the L-tartrate salt is in crystalline form, preferably form E.

15. A process according to claim 14 which comprises refluxing said compound of formula [I] in ethanol and adding dropwise thereto a solution of L-tartaric acid in a mixture of water and ethanol.

16. A process according to claim 15 wherein the ratio of ethanol:water in the final mixture after addition of the L-tartaric acid solution is at least about 15:1, more preferably about 37.5:1.

17. A process according to claim 15 or claim 16 which comprises maintaining the temperature at 75 to 78° C. during the addition of the solution of L-tartaric acid.

18. A process according to any one of claims 1 to 17 which further comprises the step of preparing compound [III], where R₂ is H, by: 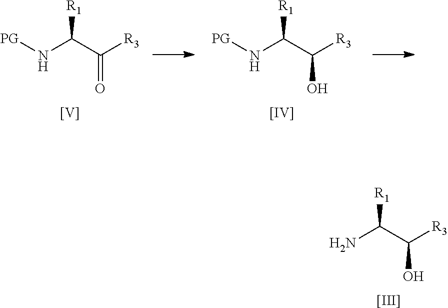
(a) treating a compound [V] with (S)-2-Me-CBS-oxazoborolidine and borane-N,N-diethylaniline complex in a solvent comprising THF to form a compound [IV]; and
(b) removing the protecting group PG from said compound [IV] to give compound [III],
wherein PG is a protecting group, preferably Boc, R¹ is alkyl or haloalkyl, and R³ is alkyl, haloalkyl or aryl.

19. A process according to claim 18 wherein step (b) comprises treating said compound [IV] with gaseous HCl in methanol, concentrating in vacuo, dissolving in ethyl acetate and then sparging with NH₃.

20. A process according to any preceding claim for preparing a compound of formula [1], or a pharmaceutically acceptable salt thereof: 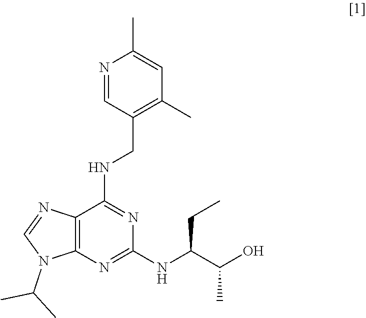 [SMILES: CC[C@H](Nc1nc(NCc2cnc(C)cc2C)c2ncn(C(C)C)c2n1)[C@@H](C)O]
said process comprising the steps of: 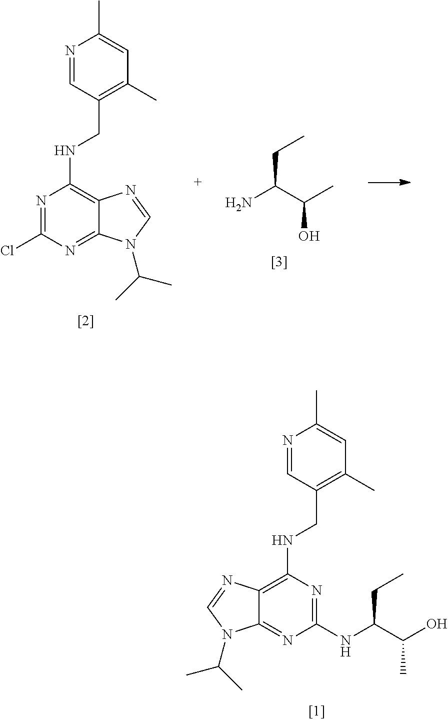
(i) forming a reaction mixture comprising a compound of formula [2], and a compound of formula [3];
(ii) heating said reaction mixture to a temperature of at least about 130° C. to form a compound of formula [1];
(iii) isolating said compound of formula [1] from the mixture and optionally recovering unreacted compound of formula [3]; and
(iv) optionally converting said compound of formula [1] into salt form.

21. A process according to claim 20 which further comprises the step of preparing a compound of formula [2] by: 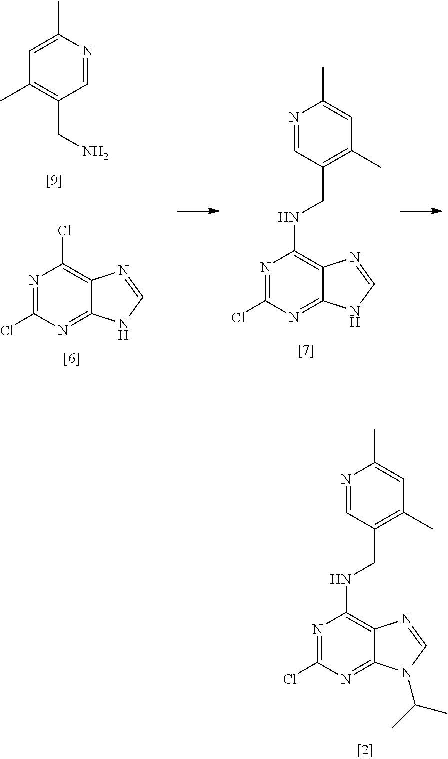
(i) treating a compound of formula [6] with a compound of formula [9], or a salt thereof, to form a compound of formula [7]; and
(ii) treating said compound of formula [7] with isopropyl bromide to form a compound of formula [2].

22. A process according to claim 20 which further comprises the step of preparing a compound of formula [2] by: 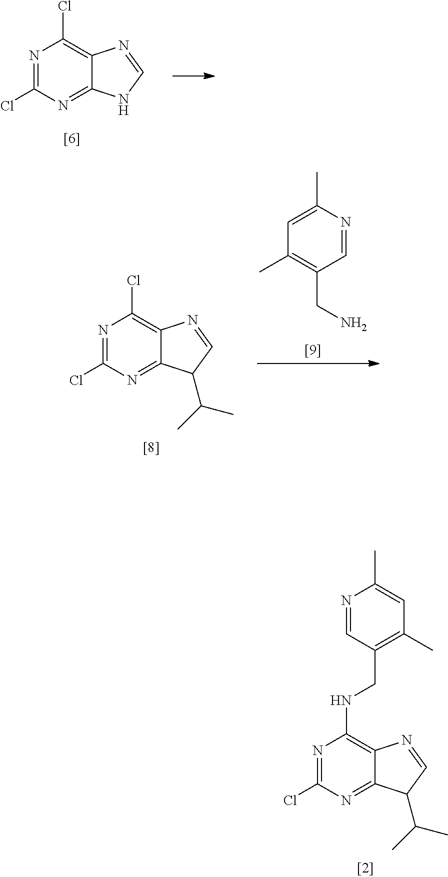
(i) treating a compound of formula [6] with isopropyl bromide to form a compound of formula [8]; and
(ii) treating said compound of formula [8] with a compound of formula [9], or a salt thereof, to form a compound of formula [2].

23. A process according to claim 21 or claim 22, wherein step (ii) of claim 21 or step (i) of claim 22 is carried out in DMSO in the presence of K₂CO₃.

24. A process according to claim 21 or claim 22, wherein step (i) of claim 21 or step (ii) of claim 22 is carried out in ⁿBuOH in the presence of a base, preferably, DIPEA.

25. A process according to any one of claims 20 to 22 wherein compound [3] has a diastereomeric excess of at least 85%, more preferably, at least 90%, even more preferably, at least 95%.

26. A process according to any one of claims 20 to 25 which further comprises the step of preparing a compound of formula [3] by: 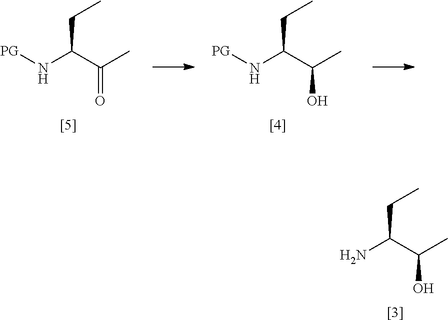
(a) treating a compound of formula [5] with (S)-2-Me-CBS-oxazoborolidine and borane-N,N-diethylaniline complex in a solvent comprising THF to form a compound of formula [4]; and
(b) removing the protecting group PG from said compound of formula [4] to give a compound of formula [3],
where PG is a protecting group, preferably Boc.

27. A process according to any one of claims 20 to 26 which comprises refluxing the product isolated in step (iii) of claim 20 in ethanol and adding dropwise thereto a solution of L-tartaric acid in a mixture of water and ethanol.

28. A process according to claim 27 which further comprises the step of polish filtering the mixture, warming the filtrate to a temperature of about 60 to about 65° C. and seeding with crystalline [1]-L-tartrate form E.

29. A process according to claim 28 which comprises stirring the seeded filtrate at a temperature of about 60 to about 65° C. for at least 1 hour.

30. A process according to claim 29 which further comprises the step of cooling the mixture to a temperature of about 15 to about 20° C. and stirring at that temperature for at least 1 hour to induce crystallisation of compound [1]-L-tartrate.

31. A process according to claim 30 wherein the cooling rate is about 5 to about 10° C./hour.

32. A process according to any one of claim 30 or 31 wherein the compound [1]-L-tartrate is filtered, washed with ethanol and dried in vacuo.

33. A process for preparing a compound of formula [I], or a pharmaceutically acceptable salt thereof, 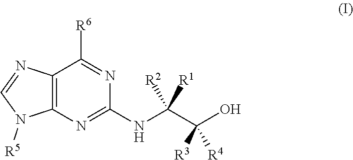
wherein R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and 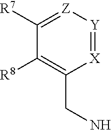
where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of: 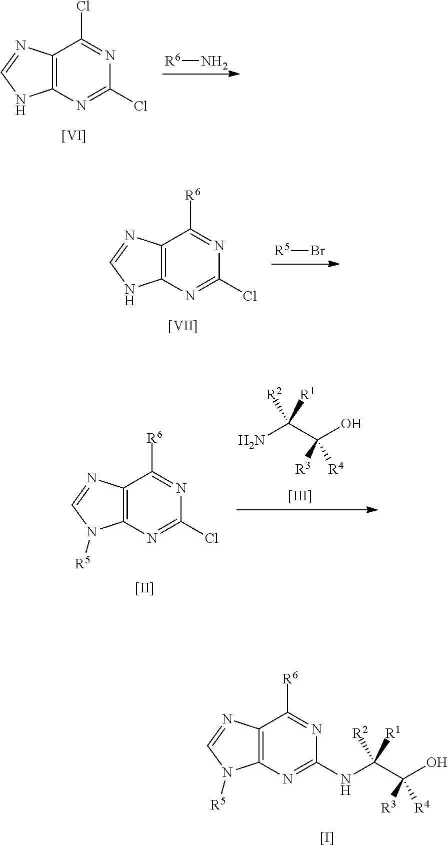
(a) treating a compound of formula [VI] with R⁶—NH₂ or a salt thereof to form a compound of formula [VII];
(b) treating said compound of formula [VII] with R⁵Br to form a compound of formula [II];
(c) forming a reaction mixture comprising a compound of formula [II], and a compound of formula [III] and heating said reaction mixture to a temperature of at least about 130° C. to form a compound of formula [I];
(d) isolating said compound of formula [I] from the mixture and optionally recovering unreacted compound of formula [III]; and
(e) optionally converting said compound of formula [I] into salt form.

34. A process of preparing a compound of formula [1], or a pharmaceutically acceptable salt thereof, said process comprising the steps of: 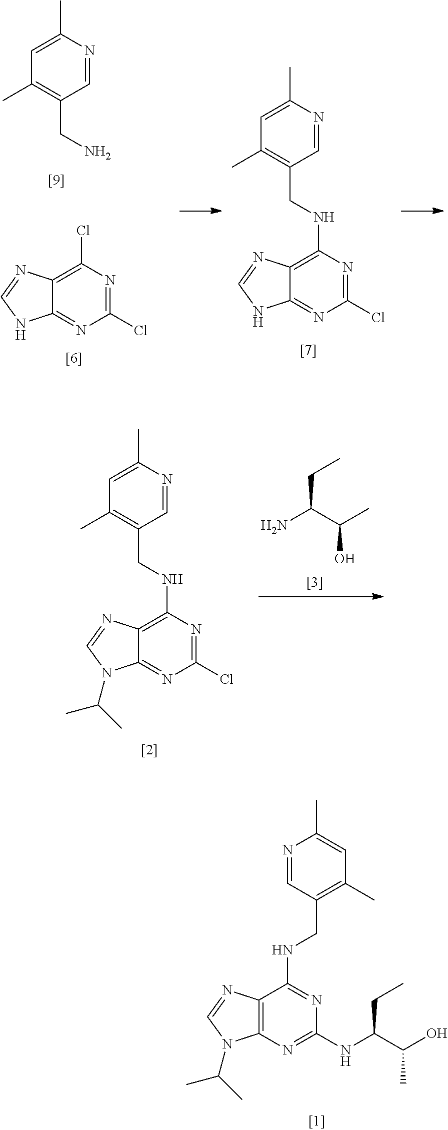
(a) treating a compound of formula [6] with a compound of formula [9], or a salt thereof, to form a compound of formula [7];
(b) treating said compound of formula [7] with isopropyl bromide to form a compound of formula [2];
(c) forming a reaction mixture comprising a compound of formula [2], and compound of formula [3] and heating said reaction mixture to a temperature of at least about 130° C. to form a compound of formula [1];
(d) isolating said compound of formula [1] from the mixture and optionally recovering unreacted compound of formula [3]; and
(e) optionally converting said compound of formula [1] into salt form.

35. A process for preparing a compound of formula [I], or a pharmaceutically acceptable salt thereof, 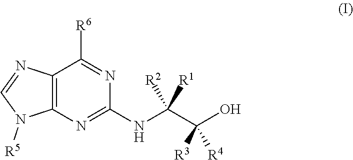
wherein:
R¹ and R² are each independently H, alkyl or haloalkyl;
R³ and R⁴ are each independently H, alkyl, haloalkyl or aryl;
R⁵ is alkyl, alkenyl, cycloalkyl or cycloalkyl-alkyl, each of which may be optionally substituted with one or more OH groups;
R⁶ is selected from cyclopropylamino, cyclopropylmethylamino, cyclobutylamino, cyclobutylmethylamino and 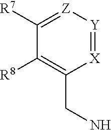
where one of X, Y and Z is N and the remainder are CR⁹;
R⁷, R⁸ and each R⁹ are independently H, alkyl or haloalkyl, wherein at least one of R⁷, R⁸ and R⁹ is other than H;
said process comprising the steps of: 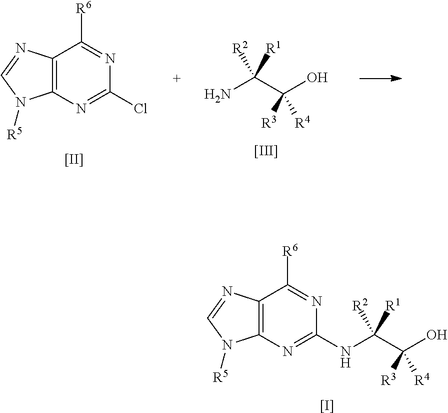
(i) forming a reaction mixture comprising a compound of formula [II], and a compound of formula [III];
(ii) heating said reaction mixture to a temperature of at least about 130° C. to form a compound of formula [I];
(iii) optionally isolating said compound of formula [I] from the mixture and optionally recovering unreacted compound of formula [III]; and
(iv) optionally converting said compound of formula [I] into salt form.
